SEQUENCE LISTING (2 sequences)

SEQ ID NO: 1 - DNA, 9654 bp, synthetic construct
  source 1..9654: mol_type = other DNA
caacttaatatcatcggtaggccaaattgagcagcccactcagtcgtcgaagctgattcagccaccacataaaccggtgcgccacctctgctatacgccgcggggtttacttttaccttatggaacttgatatgttcattatcagcttccatatatccctctgtcatgccattctttatcagcccgtaccagcattccgctaaggcgcgactgttattcatatctgtgccgaatacgcgaaagtccttgttgtaaagccctcggcaaataccaaaccgaaatcgtccttttgacatttgatccaataaattcacatcttcaagttggcgtactggatgggctgtgggaagaacaatagcggcagttcctacattcaattttttagtcgcgccaagtaaatatgcagcagcgacataagggttaccaagcaaaccaaactccgtgaaatgatgctccagtaaccatacggtatcaaaaccacactcctcagagatgcgacctaatttaaccaaacgtttcattacctctgtttgagaaaattggggaggttggtatgtaagcaaaaagtttccaaatttcatagagagtccttatattgctatttgagtgatagaatatctcaatagattttaagacagagaaattgcttgattttcaatctcaattctcattcggcgttcattgactgtcgcaatagttaaatgttcaaatgacggttcagtaatatcaacatcaatatccagatgatcattatccatcgcgatagcggctttcgtaaccgattgataaaaattgcgcaggaccactaaattttcactcaagtcatgcgaacttcctaacaaagaatatatcttgcatcgattactacgaatatttgataacaatgtgataacttcatcttgcttgacccaattatcgttatttgcagtaaaagcaataaacggtatatcaagatacatcatgttattaattgtagaagctaaatcttcccaaccaaaatcaagacaatctctcgcaaagacttcagcacccaatttatggccttcaaaatctagattattcggcaattcattaatgggtagactgagataatcaaaccctaaagctctttcaagagaatatcttaagttaacaacaccgactgcggtgattaaaaacgaagcattgatttcagataggcttgcataagctatccgcgcagataagcttgaagccaacataccgaagttatttatttttcgtgtagttaaccaatcaaccactgctaacaagctctgctttcctatagacattgtaaattcatcaattgtccctgaactcaatccaacgtggtgaagcgaatcatagcggatcacatgaaatccattccgcgataaatattccgccagaccagcaaaatgatccatcctgcgggcaaaaccagacgcaataataatggcattctttctctttgggctgttttcttctggcagcgtttcccaaacatgaatttttttatttccttcaacacaaataacgtggtcgatggttttatattttgattcattttccatactttttacctattatgggacaaatacaaggaacttatcttcttccaggaatcgagtctgttctatttcaaccgcaacatccttagccgtatagttagatggcctttcatgagaaatatatgtcactaatcgttgcaacggtctcattccgtcatgagatccaccaactcgaaatatgttattcattcctgcttctacaatcctttccgcaccttttaatgctaacgcatctcgatatttaaatgatgactcccaaggaaaaatagatatggtttgcgtcttatttttttgaacataaggcaatatttgctcaatattatcgacgtgatgaaggtacacacatctgccaagtggttgattaaattccacacctgcatttgactcaataatcatccaacgttgatgaatatccacctctacttttaatccagcaaacaagctttctttttgaactaaagaataggccgccttttcatcaaaatcttttttggcattcggtaatatatgcgcatatagattaagtttttctatcaacgctaacttaaattcctcataatgatttcccatgtaatatatgttttgggcagaaaaacaagctcgctgatcgtaaaaacaaacatcatgagccgcacctgtcgctgcggacgtcaaatcaacaggattatcgataatgcaaagactctttttagaaccaaatttaatcacatcagcataagatggcgcatgctctaccgcccaattaatcgcatctggccctccccaagcgacaataacatccgcatgttgcataatttcttttgcgagtgatgtatcaccttggtggggccaatatataacagataaagagcgcgttatcggatgattagggtctacatcaataaaacttaacgctaatgcattagcggtaaaaggatcggttgacgatgtttttataatacactgattcttagttaaaattgcgcgtaatatagacatgatcccagataatggaacattacctgccaacagatgtacagatttacctttcggaaaagcccgaacataactttcatcctgaggtagccattcatccatgatatggcgagaaccaagttcattttctacaacatcataaaggccgcctttagaacataaaatcatagatatccaattggcctctagcttagccatttcttctgaatatcccatatatttttttaagtcacgaatgtatgtcctgcgtcttgagtattcttcatttttccatctttgccctaccgtatagagaaaattgacaatgttatgcaaccgtaattcgttatttccattacaatcaataatgttttttacatgagagtcattcaatattggcaggtaaacactattatcaccaaaattaatggattgcactaaatcatcaccttcgggaaagatttcaacctggccgttaataatgaatgaaatttttttagtcatcatgaggattggcgtcctttgtcgttggctgcatgagaaaccgccccgggagcgctccttgatcggcgggcggggaaaatctaggcttgcgcaggatttgcggcaaacgacgaatactcgcggcagccattagaaaaatttggagcagagattggttcgaccccatttgccgctgaacgccctgcgcgccttcgaagcttcggcccggcacctgagcttcacccgcgcggccatcgagctgtgcgtgacccaggcggcggtcagccaccaggtgaagagcctcgaggagcgtctcggcgtggccctgttcaagcgtctgccgcgcggcctcatgctgacccacgagggcgagagcctgctgccggtgctgtgtgactccttcgaccgcatcgctggcctgctggaacgcttcgagggtggccactaccgggacgtgctcaccgtcggcgcggtcggaaccttcacggtcggttggctgctgccgcggctggaggacttccgggcgcgccatcccttcatcgatctgcgcctgtccacccacaacaaccgcgtcgacatcgccgccgaggggctcgactacgcgatccgcttcggcggcggcgcctggcacggcaccgaggcgctggcgttgttcgaggcgccgctgacggtgctctgctgcccggaggtcgccgcccagttgcacagtcccgccgacctgctgcagcacaccctgctgcgctcctaccgcgccgacgagtggccgctgtggttccaggcggccggactgccggcgcacgcgccgctgacccggagcatcgtctttgacacctcgctggccatgctcgaggcggcccgccagggtgtcggcgtggccctggcgccggcggcgatgttcgcccggcaactggccagcgagagcatccggcgtccgttcgccaccgaagtgagtaccggcagctactggctgacgcgcttgcagtcgcggggggagaccagcgcgatgctggtgttcagggagtggttgctgggccaggccgtcttggagagcgaggcgtagagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccccgggtaccgagctcccagtcacgacgttgtaaaacgacggccagtgagcgcgcgtaatacgactcactatagggcgaattggagctccaccgcggtggcggccgctctagaactagtggatcccccgggctgcaggtcatcaactatcaaacgcttcggttaagcttaaagcacaccctttctgcgtcctcgtattgacgcgacgtaaaatttcaacgagcacgccgggatacttaccatattctctgctaattatcccgacatcatcggtaacaataaatgctggataactggttgctgacgcatccatataactcatcaaccccggcgttccatcaggtacaggtttcaacgtttcaggatcaagcgctcgcgcatatacccacggcggaacatgtttacgctgcatttcatcctcaaagaaacaagtgttgagttcaacttgattaaatatatctcggatctgactaatatcactgagattgaaagtatcaaataaaagatgattgaaatcatcacgtttcagagattctttttcgtaacttttccagccgcctccggttatgatataaaggcttttatctccagaaaatgagatttttttatctttcatataatggcagagtaaataaataaagtatggcgaaccaataagacaaagatctttcccttgattttttattcgttcaagactattcaatgttttaacaaaatctattcgttcttctgttacggtaaatgtcgtaggatataacaattccaccaaactcataacatatttaaaccaaatattatgagcattaaatctatctggtcccaaattgactaattctatttgatgatcaaaccaactaccaacatatttcatgccataactcacagagcctaagagtctctcaatacttaatctgtcacgcgccacctgactttttaaaccattcgtgccgctactggtaaaccaactttcaatctcgttttcctgagaagttaataagcgagtaaacttaaaaaccgatgttgggaatacaggtatgtcatcaatttccgtaatattgtcatctactttgtgtgcctgacagtagtgacgatattctcgacaatgtttataatgattacgaaatgcatcaagcacaagtttctttctgattttttcctgctcgtcgtaagaccacactaatggatcgctcgaaaaaatcaaatcatcaatttctgagcttgctgtaatttcttgtttatcaacatatgaagtcatacctgttttactcctcaagataatattagaaagtatggcagcactgctgtcatactcttttatacccttcatctttcaagctgctgctttgttggctgctttcactcaccccagtcacatagttatctatgctcctggggattcgttcacttgccgcctcgctgcaactcgaaatctattaggtatattccatgtggtacttcttaatattatcatcaacaatattgattacatttttttggctcatcaaatcattcattggttcaaaggacagcaatacacttttcgcaccacacttttcaattgccaacttagccgcagttatacactccgtataatttccgacagcgttttctgcaattatttcttcaagtttattttcgaaattttcattagggtgcatttcaagaacataatcactaataaatgcacgcgtctcttgtttagctttattactatcttcgttatagttaactaatatcattaactgatggtctatctctgataggtcaacgtcatatttatccgcaacggctttatatctttcagcatattcatatctaacatcattagaatcatcccacttaaagatgagaggaataccttttttggccgcccactcaacaatatgatgactggttgctgttacatatttccgaggtccgcctggcgtataagcatggggatttacagatattttagggaagctataaaaatcgttatctggattacaatagcctgttgttaaagcatcgttaatgatttcataacactcttcaaatagttgctgttgatattcaaccgggcgattaaaaaaatgcatttcatcttttttttcgcaatcactaaaccctaaaataaatctcccttcacttaactgatccaataagcaagcttcctccgctatgcggacaggatgatgagttgtaatgatgtgatttaatgaaccaattttaattttctctgttaaaccgagcagaaaaccagaaacagtcagaggagcgccgacaacaccattatctgaaaaatgattttcatacactaaaatctgttcaaaattcaacttatcaacatactccgttatttcctgcatgcgaactatactttgttcttgaacagttgttgaattgatgaagttaaggaagaacaatccaaatttcatttctttctccttagctaatataatagcgaacgttgtttttctttaagaaatggcatgacatcagactggaagagcttcatggaagcaataatttcgtctactgttccattagcttcaaatccacaacaaatatttgatattcctgtagcatcaatgtctttttgaattatgtcaatacattcctgcggcgttcccacgggattgatttcgtaactgtaatcaatacggcgattagtatctttatgtccttttaatacaaagtcacgccactgccctttattgaaatcataacctcttgtttggtctgaatcatcaaaaatagtcgtagcattcacataagaatcataccaatgccccagaaatttccggcaaatctctttcgctttaattgagtcatgatctacagatgttatatatgataagcaatggtcgatattatgaatatcgtgcccatattcttgagccacttcattataaagctcaagttgtgctttcttttcgttagtatttataatc

SEQ ID NO: 2 - DNA, 10558 bp, synthetic construct
  source 1..10558: mol_type = other DNA
tcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctctacgcctcgctctccaagacggcctggcccagcaaccactccctgaacaccagcatcgcgctggtctccccccgcgactgcaagcgcgtcagccagtagctgccggtactcacttcggtggcgaacggacgccggatgctctcgctggccagttgccgggcgaacatcgccgccggcgccagggccacgccgacaccctggcgggccgcctcgagcatggccagcgaggtgtcaaagacgatgctccgggtcagcggcgcgtgcgccggcagtccggccgcctggaaccacagcggccactcgtcggcgcggtaggagcgcagcagggtgtgctgcagcaggtcggcgggactgtgcaactgggcggcgacctccgggcagcagagcaccgtcagcggcgcctcgaacaacgccagcgcctcggtgccgtgccgggcgccgccgccgaagcggatcgcgtagtcgagcccctcggcggcgatgtcgacgcggttgttgtgggtggacaggcgcagatcgatgaagggatggcgcgcctggaagtcctccagccgcggcagcagccaaccgaccgtgaaggttccgaccgcgccgacggtgagcacgtcccggtagtggccaccctcgaagcgttccagcaggccagcgatgcggtcgaaggagtcacacagcaccggcagcaggctctcgccctcgtgggtcagcatgaggccgcgcggcagacgcttgaacagggccacgccgagacgctcctcgaggctcttcacctggtggctgaccgccgcctgggtcacgcacagctcgatggccgcgcgggtgaagctcaggtgccgggccgaagcttcgaaggcgcgcagggcgttcagcggcaaatggggtcgaaccaatctctgctccaaatttttctaatggctgccgcgagtattcgtcgtttgccgcaaatcctgcgcaagcctagattttccccgcccgccgatcaaggagcgctcccggggcggtttctcatgcagccaacgacaaaggacgccaatcctcatgatgactaaaaaaatttcattcattattaacggccaggttgaaatctttcccgaaggtgatgatttagtgcaatccattaattttggtgataatagtgtttacctgccaatattgaatgactctcatgtaaaaaacattattgattgtaatggaaataacgaattacggttgcataacattgtcaattttctctatacggtagggcaaagatggaaaaatgaagaatactcaagacgcaggacatacattcgtgacttaaaaaaatatatgggatattcagaagaaatggctaagctagaggccaattggatatctatgattttatgttctaaaggcggcctttatgatgttgtagaaaatgaacttggttctcgccatatcatggatgaatggctacctcaggatgaaagttatgttcgggcttttccgaaaggtaaatctgtacatctgttggcaggtaatgttccattatctgggatcatgtctatattacgcgcaattttaactaagaatcagtgtattataaaaacatcgtcaaccgatccttttaccgctaatgcattagcgttaagttttattgatgtagaccctaatcatccgataacgcgctctttatctgttatatattggccccaccaaggtgatacatcactcgcaaaagaaattatgcaacatgcggatgttattgtcgcttggggagggccagatgcgattaattgggcggtagagcatgcgccatcttatgctgatgtgattaaatttggttctaaaaagagtctttgcattatcgataatcctgttgatttgacgtccgcagcgacaggtgcggctcatgatgtttgtttttacgatcagcgagcttgtttttctgcccaaaacatatattacatgggaaatcattatgaggaatttaagttagcgttgatagaaaaacttaatctatatgcgcatatattaccgaatgccaaaaaagattttgatgaaaaggcggcctattctttagttcaaaaagaaagcttgtttgctggattaaaagtagaggtggatattcatcaacgttggatgattattgagtcaaatgcaggtgtggaatttaatcaaccacttggcagatgtgtgtaccttcatcacgtcgataatattgagcaaatattgccttatgttcaaaaaaataagacgcaaaccatatctatttttccttgggagtcatcatttaaatatcgagatgcgttagcattaaaaggtgcggaaaggattgtagaagcaggaatgaataacatatttcgagttggtggatctcatgacggaatgagaccgttgcaacgattagtgacatatatttctcatgaaaggccatctaactatacggctaaggatgttgcggttgaaatagaacagactcgattcctggaagaagataagttccttgtatttgtcccataataggtaaaaagtatggaaaatgaatcaaaatataaaaccatcgaccacgttatttgtgttgaaggaaataaaaaaattcatgtttgggaaacgctgccagaagaaaacagcccaaagagaaagaatgccattattattgcgtctggttttgcccgcaggatggatcattttgctggtctggcggaatatttatcgcggaatggatttcatgtgatccgctatgattcgcttcaccacgttggattgagttcagggacaattgatgaatttacaatgtctataggaaagcagagcttgttagcagtggttgattggttaactacacgaaaaataaataacttcggtatgttggcttcaagcttatctgcgcggatagcttatgcaagcctatctgaaatcaatgcttcgtttttaatcaccgcagtcggtgttgttaacttaagatattctcttgaaagagctttagggtttgattatctcagtctacccattaatgaattgccgaataatctagattttgaaggccataaattgggtgctgaagtctttgcgagagattgtcttgattttggttgggaagatttagcttctacaattaataacatgatgtatcttgatataccgtttattgcttttactgcaaataacgataattgggtcaagcaagatgaagttatcacattgttatcaaatattcgtagtaatcgatgcaagatatattctttgttaggaagttcgcatgacttgagtgaaaatttagtggtcctgcgcaatttttatcaatcggttacgaaagccgctatcgcgatggataatgatcatctggatattgatgttgatattactgaaccgtcatttgaacatttaactattgcgacagtcaatgaacgccgaatgagaattgagattgaaaatcaagcaatttctctgtcttaaaatctattgagatattctatcactcaaatagcaatataaggactctctatgaaatttggaaactttttgcttacataccaacctccccaattttctcaaacagaggtaatgaaacgtttggttaaattaggtcgcatctctgaggagtgtggttttgataccgtatggttactggagcatcatttcacggagtttggtttgcttggtaacccttatgtcgctgctgcatatttacttggcgcgactaaaaaattgaatgtaggaactgccgctattgttcttcccacagcccatccagtacgccaacttgaagatgtgaatttattggatcaaatgtcaaaaggacgatttcggtttggtatttgccgagggctttacaacaaggactttcgcgtattcggcacagatatgaataacagtcgcgccttagcggaatgctggtacgggctgataaagaatggcatgacagagggatatatggaagctgataatgaacatatcaagttccataaggtaaaagtaaaccccgcggcgtatagcagaggtggcgcaccggtttatgtggtggctgaatcagcttcgacgactgagtgggctgctcaatttggcctaccgatgatattaagttggattataaatactaacgaaaagaaagcacaacttgagctttataatgaagtggctcaagaatatgggcacgatattcataatatcgaccattgcttatcatatataacatctgtagatcatgactcaattaaagcgaaagagatttgccggaaatttctggggcattggtatgattcttatgtgaatgctacgactatttttgatgattcagaccaaacaagaggttatgatttcaataaagggcagtggcgtgactttgtattaaaaggacataaagatactaatcgccgtattgattacagttacgaaatcaatcccgtgggaacgccgcaggaatgtattgacataattcaaaaagacattgatgctacaggaatatcaaatatttgttgtggatttgaagctaatggaacagtagacgaaattattgcttccatgaagctcttccagtctgatgtcatgccatttcttaaagaaaaacaacgttcgctattatattagctaaggagaaagaaatgaaatttggattgttcttccttaacttcatcaattcaacaactgttcaagaacaaagtatagttcgcatgcaggaaataacggagtatgttgataagttgaattttgaacagattttagtgtatgaaaatcatttttcagataatggtgttgtcggcgctcctctgactgtttctggttttctgctcggtttaacagagaaaattaaaattggttcattaaatcacatcattacaactcatcatcctgtccgcatagcggaggaagcttgcttattggatcagttaagtgaagggagatttattttagggtttagtgattgcgaaaaaaaagatgaaatgcatttttttaatcgcccggttgaatatcaacagcaactatttgaagagtgttatgaaatcattaacgatgctttaacaacaggctattgtaatccagataacgatttttatagcttccctaaaatatctgtaaatccccatgcttatacgccaggcggacctcggaaatatgtaacagcaaccagtcatcatattgttgagtgggcggccaaaaaaggtattcctctcatctttaagtgggatgattctaatgatgttagatatgaatatgctgaaagatataaagccgttgcggataaatatgacgttgacctatcagagatagaccatcagttaatgatattagttaactataacgaagatagtaataaagctaaacaagagacgcgtgcatttattagtgattatgttcttgaaatgcaccctaatgaaaatttcgaaaataaacttgaagaaataattgcagaaaacgctgtcggaaattatacggagtgtataactgcggctaagttggcaattgaaaagtgtggtgcgaaaagtgtattgctgtcctttgaaccaatgaatgatttgatgagccaaaaaaatgtaatcaatattgttgatgataatattaagaagtaccacatggaatatacctaatagatttcgagttgcagcgaggcggcaagtgaacgaatccccaggagcatagataactatgtgactggggtgagtgaaagcagccaacaaagcagcagcttgaaagatgaagggtataaaagagtatgacagcagtgctgccatactttctaatattatcttgaggagtaaaacaggtatgacttcatatgttgataaacaagaaattacagcaagctcagaaattgatgatttgattttttcgagcgatccattagtgtggtcttacgacgagcaggaaaaaatcagaaagaaacttgtgcttgatgcatttcgtaatcattataaacattgtcgagaatatcgtcactactgtcaggcacacaaagtagatgacaatattacggaaattgatgacatacctgtattcccaacatcggtttttaagtttactcgcttattaacttctcaggaaaacgagattgaaagttggtttaccagtagcggcacgaatggtttaaaaagtcaggtggcgcgtgacagattaagtattgagagactcttaggctctgtgagttatggcatgaaatatgttggtagttggtttgatcatcaaatagaattagtcaatttgggaccagatagatttaatgctcataatatttggtttaaatatgttatgagtttggtggaattgttatatcctacgacatttaccgtaacagaagaacgaatagattttgttaaaacattgaatagtcttgaacgaataaaaaatcaagggaaagatctttgtcttattggttcgccatactttatttatttactctgccattatatgaaagataaaaaaatctcattttctggagataaaagcctttatatcataaccggaggcggctggaaaagttacgaaaaagaatctctgaaacgtgatgatttcaatcatcttttatttgatactttcaatctcagtgatattagtcagatccgagatatatttaatcaagttgaactcaacacttgtttctttgaggatgaaatgcagcgtaaacatgttccgccgtgggtatatgcgcgagcgcttgatcctgaaacgttgaaacctgtacctgatggaacgccggggttgatgagttatatggatgcgtcagcaaccagttatccagcatttattgttaccgatgatgtcgggataattagcagagaatatggtaagtatcccggcgtgctcgttgaaattttacgtcgcgtcaatacgaggacgcagaaagggtgtgctttaagcttaaccgaagcgtttgatagttgatgacctgcagcccgggggatccactagttctagagcggccgccaccgcggtggagctccaattcgccctatagtgagtcgtattacgcgcgctcactggccgtcgttttacaacgtcgtgactgggagctcggtacccggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagttctctgatgttacattgcacaagataaaaatatatcatcatgaacaataaaactgtctgcttacataaacagtaatacaaggggtgttatgagccatattcaacgggaaacgtcttgctcgaagccgcgattaaattccaacatggatgctgatttatatgggtataaatgggctcgcgataatgtcgggcaa

(19) United States
(12) Patent Application Publication
(10) Pub. No.: US 2026/0117324 A1
(43) Pub. Date: Apr. 30, 2026

(54) Biosensor and Assays for Rapid Detection of Antibiotic Resistance

(71) Applicant: Queen's University at Kingston, Kingston (CA)

(72) Inventors: Mitchell Jeffs, Kingston (CA); Christopher Lohans, Kingston (CA)

(21) Appl. No.: 19/278,295

(22) Filed: Jul. 23, 2025

Related U.S. Application Data
(60) Provisional application No. 63/674,895, filed on Jul. 24, 2024.

(51) Int. Cl. C12Q 1/6897; C12N 1/20; C12N 9/99; C12N 15/70; C12Q 1/02; C12Q 1/34; C12Q 1/66; C12R 1/19

(52) U.S. Cl. CPC C12Q 1/6897; C12N 1/20; C12N 9/99; C12N 15/70; C12Q 1/025; C12Q 1/34; C12Q 1/66; C12R 2001/19; G01N 2333/90241; G01N 2333/986

(57) ABSTRACT

A plasmid includes a reporter and an inducible promoter that enables transcription of the reporter in the presence of one or more products of β-lactam antibiotic activity, wherein transcription of the reporter produces a signal. The plasmid may be incorporated into an assay to detect β-lactamase activity by reducing the signal in the presence of the β-lactamase, or β-lactamase inhibition by producing the signal in the presence of the β-lactamase inhibitor. A cell may be transformed with the plasmid to provide a biosensor cell for assays and kits used to detect, assess, and/or characterize activity of a compound as a β-lactamase inhibitor, or to detect, assess, and/or characterize a β-lactamase producing organism.

### RELATED APPLICATION

This application claims the benefit of the filing date of Application No. 63/674,895, filed Jul. 24, 2024, the contents of which are incorporated herein by reference in their entirety.

### SEQUENCE LISTING STATEMENT

A computer readable form of the Sequence Listing is filed with this application by electronic submission and is incorporated into this application by reference in its entirety. The Sequence Listing is contained in the file created on Jul. 24, 2025 having the file name 2024-004-03US_V4.xml and is 23,000 bytes in size.

### FIELD

The invention relates generally to the detection of antibiotic resistance. More specifically, the invention relates to genetic constructs, biosensor cells, and assays for detecting and quantification of β-lactamase activity and inhibition, and for detecting β-lactamase-producing bacteria.

### BACKGROUND

β-lactams are currently the most commonly prescribed class of antibiotics worldwide, and include the penicillins, cephalosporins, carbapenems and monobactams. Several drugs belonging to this class (e.g., carbapenems such as meropenem, ertapenem and imipenem) are considered last-resort antibiotics for targeting multi-drug resistant pathogens. Carbapenems are used for a wide array of clinical indications including urinary tract infections, pneumonia, intra-abdominal infections and meningitis, among others.

Due to the widespread use of carbapenems, the global emergence and increasing prevalence of carbapenemase-producing organisms (CPOs) poses a significant global health concern. These enzymes confer bacteria with resistance to carbapenems (as well as other β-lactams) by hydrolyzing the core β-lactam ring of these antibiotics. Carbapenemases are a type of β-lactamase, a diverse group of hydrolytic enzymes which also include penicillinases (which hydrolyze penicillins) and extended spectrum β-lactamases (ESBLs; hydrolyze penicillins and cephalosporins). Like other β-lactamases, carbapenemases are further broken down into several classes based on the Ambler classification system: class A (e.g., KPCs) and class D (e.g., OXAs) enzymes rely on an active site serine residue to catalyze carbapenem hydrolysis, while class B enzymes (e.g., NDMs, IMPs, VIMs) utilize zinc ions to facilitate hydrolysis. These enzymes are produced by many bacterial pathogens, such as Escherichia coli, Pseudomonas aeruginosa, Acinetobacter baumannii, Klebsiella pneumoniae, and Enterobacter cloacae, and infections caused by these CPOs are associated with increased mortality rates. Thus, the identification of inhibitors that prevent carbapenemases and other β-lactamases from degrading β-lactams is of great importance. Furthermore, the rapid detection of CPOs in clinical settings is vital to ensure administration of timely and appropriate antimicrobial therapy, and prevention and containment of outbreaks caused by these pathogens.

Currently, several different assays are employed for the detection of CPOs. Traditional phenotypic tests approved by the Clinical and Laboratory Standards Institute (CLSI) include growth-based assays such as the modified carbapenem inactivation method (mCIM). Although the mCIM is widely employed in hospital settings due to its low cost per test and ease of use, this assay suffers from long turn-around times, typically requiring 18-24 h (not including the time needed for CPO isolation and culturing) before results are obtained [1, 2]. To address this limitation, rapid phenotypic tests have also been developed. Currently the sole rapid carbapenemase activity assay endorsed by the CLSI is the CARBA-NP test, which significantly reduces turnaround time compared to the mCIM with a time-to-positivity of ≤2 hours [3, 4]. However, some laboratories have observed that this assay suffers from lower sensitivity for CPOs that produce weak carbapenemases, such as the OXA-48-like β-lactamases [5-9]. Other rapid tests employ fluorogenic or chromogenic β-lactams [10-12]; however, their use may be limited due to costs associated with the synthesis or purchase of these non-clinical substrates, and these tests have not yet been approved by the CLSI. In recent years, the development of molecular tests has increased due to their high sensitivities and quick turn-around times. Several polymerase chain reaction (PCR) panels have been applied to the detection of carbapenemase genes [13-16]. Despite these advances, usage of genotypic assays is limited in lower resource settings due to high costs associated with equipment and reagents, the need for highly trained staff [17], and potential limitations associated with the detection of novel carbapenemase variants. Consequently, there is a need for rapid, sensitive, and inexpensive tests for measuring the activity and inhibition of carbapenemases and other β-lactamases, and for detecting CPOs.

### SUMMARY

According to one aspect of the invention there is provided a plasmid comprising: a reporter; an inducible promoter; wherein the inducible promoter enables transcription of the reporter in the presence of one or more products of β-lactam antibiotic activity; wherein transcription of the reporter produces a signal; wherein a greater amount of signal is produced in the presence of a β-lactamase inhibitor relative to an amount of signal produced upon degradation of a β-lactam antibiotic by a β-lactamase. In some embodiments the plasmid may be incorporated into an assay to detect: β-lactamase activity by reducing the signal in the presence of the β-lactamase; or β-lactamase inhibition by producing the signal in the presence of the β-lactamase inhibitor.

In one embodiment, the β-lactamase is produced by a bacterial cell.

In one embodiment, the β-lactamase is an isolated enzyme.

In one embodiment, the reporter is a luminescent reporter and the signal is luminescence.

In one embodiment, the luminescent reporter comprises a luciferase reporter.

In one embodiment, the luciferase reporter comprises a luxCDABE operon.

In one embodiment, the inducible promoter comprises a carbapenem-inducible ampC promoter and an ampR transcription factor that controls transcription of the ampC promoter.

In one embodiment, the plasmid has the nucleotide sequence of SEQ ID NO: 1.

In one embodiment, the plasmid has the nucleotide sequence of SEQ ID NO: 2.

In some embodiments, the plasmid is incorporated into a bacterial cell exposed to a β-lactam antibiotic.

According to another aspect of the invention there is provided a cell transformed with a plasmid as described herein.

In one embodiment, the cell is a bacterial cell.

In one embodiment, the cell is a strain of E. coli.

In one embodiment, the cell is a biosensor cell.

According to another aspect of the invention there is provided a biosensor cell comprising a plasmid as described herein.

In one embodiment the biosensor cell comprises a luminescent reporter and the signal is luminescence.

According to another aspect of the invention there is provided an assay for detecting, assessing, and/or characterizing a β-lactamase inhibitor, comprising: a biosensor cell as described herein; a β-lactam antibiotic; a β-lactamase; and a suitable cellular growth medium; wherein the assay detects, assesses, and/or characterizes the β-lactamase inhibitor added to the assay according to an amount of signal produced by the biosensor cell; wherein the β-lactamase inhibitor activity is indicated by the biosensor cell producing the signal.

In one embodiment of the assay, the β-lactamase is produced by a bacterial cell.

In one embodiment of the assay, the β-lactamase is an isolated enzyme.

In one embodiment the signal is a luminescent signal.

According to another aspect of the invention there is provided an assay for detecting, assessing, and/or characterizing a β-lactamase producing organism, comprising: a biosensor cell as described herein; a β-lactam antibiotic; and a suitable cellular growth medium; and optionally comprising proteinase K; wherein the assay detects, assesses, and/or characterizes the β-lactamase producing organism added to the assay according to an amount of signal produced by the biosensor cell; wherein the β-lactamase producing organism is indicated by the biosensor cell producing low or no signal. In one embodiment the signal is luminescence.

According to another aspect of the invention there is provided a kit, comprising: a biosensor cell as described herein; optionally comprising a β-lactam antibiotic or a β-lactamase; optionally comprising proteinase K; optionally comprising a suitable cellular growth medium.

According to another aspect of the invention there is provided a method for detecting, assessing, and/or characterizing activity of a compound as a β-lactamase inhibitor, comprising: incubating a biosensor cell as described herein together with a β-lactam antibiotic and a β-lactamase in a suitable cellular growth medium; adding the compound to the cellular growth medium; wherein activity of the compound as a β-lactamase inhibitor is detected, assessed, and/or characterized according to an amount of signal produced by the biosensor cell; wherein the biosensor cell produces signal when the compound exhibits β-lactamase inhibitor activity. In one embodiment the signal is luminescence.

According to another aspect of the invention there is provided a method for detecting, assessing, and/or characterizing a β-lactamase producing organism, comprising: incubating the biosensor cell of claim 16 and a β-lactam antibiotic in a suitable cellular growth medium; adding the organism to the cellular growth medium; wherein the β-lactamase producing organism is detected, assessed, and/or characterized according to an amount of signal produced by the biosensor cell; wherein the biosensor cell produces low or no signal when the organism produces the β-lactamase. In one embodiment the signal is luminescence.

### BRIEF DESCRIPTION OF THE DRAWINGS

For a greater understanding of the invention, and to show more clearly how it may be carried into effect, embodiments will be described, by way of example, with reference to the accompanying drawings, wherein:

FIG. 1A is a diagram of a plasmid construct, according to one embodiment.

FIG. 1B shows the nucleic acid sequence of the plasmid of FIG. 1A, according to one embodiment, referred to as SEQ ID NO:1.

FIGS. 2A and 2B are diagrams of a plasmid construct, according to one embodiment.

FIG. 2C shows the nucleic acid sequence of the plasmid of FIGS. 2A and 2B, according to one embodiment, referred to as SEQ ID NO:2.

FIGS. 3A and 3B are plots showing validation results of a biosensor according to one embodiment wherein (A) shows treatment of biosensor cells with amoxicillin (AMX) or imipenem (IMI) leading to a time-dependent luminescent signal, and (B) shows dose response curves for AMX and IMI with biosensor cells, measured two hours post-antibiotic exposure. n=4, error bars indicate S.D.

FIGS. 4A-4E are plots of results of serine β-lactamase (SBL) inhibition assays using the biosensor according to one embodiment, including (A) single-point TEM-116 inhibition assays and (B) TEM-116 dose response analysis wherein TEM-116-producing E. coli were treated with amoxicillin (AMX, 20 μM) in combination with the indicated concentrations of avibactam (AVI), clavulanic acid (CLAV), or tazobactam (TZB); single-point inhibition assays for (C) KPC-2 and (D) OXA-48; and (E) KPC-2 and OXA-48 dose-response inhibition by avibactam. Luminescence readings were normalized to the sample with the greatest luminescence to determine % inhibition. n=4, error bars indicate S.D.

FIGS. 5A and 5B are plots of results of NDM-1 inhibition assays using the biosensor according to one embodiment, including (A) single point NDM-1 inhibition and (B) NDM-1 dose response analysis, wherein NDM-1-producing E. coli were treated with meropenem (MPM, 2.5 μM) in combination with the indicated concentrations of ethylenediaminetetraacetic acid (EDTA), dipicolinic acid (DPA), nitrilotriacetic acid (NTA), captopril (CAP), or embelin (EMB). Luminescence readings were normalized to the sample with the greatest luminescence to determine % inhibition. n=4, error bars indicate S.D.

FIGS. 6A and 6B are plots showing the effects of knocking out the E. coli gene ompF on TEM-116 inhibition using the biosensor according to one embodiment, including dose response analysis of (A) avibactam and (B) tazobactam in combination with amoxicillin (20 μM); previously determined dose-response curves for the wild-type (WT) strain are shown for comparison. Luminescence readings were normalized to the sample with the greatest luminescence to determine % inhibition. n=4, error bars indicate S.D.

FIGS. 7A and 7B show validation results for the CPO detection biosensor assay with E. coli lab strains expressing carbapenemases OXA-48, NDM-1, IMP-1, and KPC-2, penicillinases TEM-116 and OXA-10, and the ESBL CTX-M-15, wherein (A) shows detection scores and (B) is a comparison of detection scores for carbapenemase-producing E. coli in tests using imipenem alone or imipenem with 4 μg/mL avibactam; all detection scores were calculated by determining the fold-difference in luminescence between the carbapenemase-negative control and the test strain. All strains were tested in quadruplicate. ns=non-significant; ***: p<0.001; ****: p<0.0001.

FIGS. 8A-8C show results of limit of detection studies of the biosensor with E. coli lab strains producing the carbapenemases (A) NDM-1, (B) KPC-2, and (C) OXA-48 at cell densities ranging from 10⁷ to 10² CFU/mL, wherein detection scores were calculated by determining the fold-difference in luminescence between the carbapenemase-negative control and the test strain. All strains were tested in quadruplicate. ns=non-significant; *: p<0.05; **: p<0.01.

FIG. 9 is a plot showing the distribution of detection scores for different imipenem minimum inhibitory concentration brackets for CPO isolates.

### DETAILED DESCRIPTION OF EMBODIMENTS

In one aspect, the invention provides a DNA construct (e.g., a plasmid) including a reporter and an inducible promoter that controls transcription of the reporter. The plasmid containing the reporter and inducible promoter may be a high copy plasmid (e.g., pUC19, pHSG298) to maximize signal intensity, or a low copy plasmid (e.g., pACYC184) to minimize background noise but sacrifice signal intensity. In some embodiments, the plasmid backbone is pUC19, a high copy plasmid with an ampicillin resistance marker, or it is a kanamycin-resistant derivative of pUC19. In other embodiments, other antibiotic resistance markers may be used. The reporter may be, for example, a bioluminescent reporter, a fluorescent reporter, a chromogenic reporter, etc. Transcription of the reporter in the presence of a product of β-lactam antibiotic activity produces a signal (e.g., luminescence, fluorescence, colour change, etc.), wherein the product is produced by a cell exposed to a β-lactam.

In general, a greater amount of signal is produced in the presence of a β-lactamase inhibitor relative to an amount of signal produced upon degradation of a β-lactam antibiotic by a β-lactamase. In certain embodiments, the construct comprises a luminescent reporter. According to such embodiments, upon degradation of a β-lactam antibiotic by a β-lactamase, no luminescence or a low level of luminescence is produced. Addition of an effective amount of a β-lactamase inhibitor protects the β-lactam, restoring luminescence. To the inventors' knowledge, such a reporter system applied to the quantification of β-lactamase activity and inhibition or to the detection of β-lactamase-producing bacteria such as CPOs has not been reported in the literature.

In another aspect, the invention provides a cell transformed with a plasmid according to embodiments described herein. The cell may be, for example, a bacterial cell such as E. coli. The transformed cell may be used as a whole-cell biosensor. The reporter may be, for example, a luminescent reporter, wherein exposure of the biosensor cell to a β-lactam antibiotic produces a luminescent signal. If the β-lactam antibiotic is degraded by β-lactamase or β-lactamase-producing bacteria prior to the introduction of biosensor cells, less luminescence or no luminescence is produced by the biosensor. Addition of an effective amount of a β-lactamase inhibitor protects the β-lactam, restoring luminescence.

As described herein, a biosensor according to embodiments may be used in various tests, assays, etc., for one or more of detecting β-lactamases and their activity, that is, the hydrolysis, degradation, and/or inactivation of β-lactams, discovering and evaluating β-lactamase inhibitors, e.g., screening small molecule and peptide libraries for new β-lactamase inhibitors and penicillin-binding protein (PBP)-targeting antibiotics, and detecting β-lactamase-producing organisms in laboratory, environmental, or clinical sample matrices. Another aspect of the invention therefore relates to whole-cell biosensor assays and methods according to such uses.

In another aspect, the invention provides a kit, comprising a cell transformed with a plasmid according to embodiments described herein. The transformed cell, i.e., biosensor cell, may be provided in a stabilized form, e.g., an agar stab or lyophilized. In some embodiments the kit may include a β-lactam antibiotic or a β-lactamase. In some embodiments, the kit may include a positive control strain for carbapenemase production. In some embodiments the kit may include a suitable cellular growth medium. In some embodiments, the kit may include proteinase K, a broad spectrum protease used to protect the biosensor cells from antimicrobial proteins produced by test strains, e.g., as a powder or solution. A kit according to such embodiments may be used in accordance with methods and assays described herein for one or more of detecting β-lactamases and their activity, that is, the hydrolysis, degradation, and/or inactivation of β-lactams, discovering and evaluating β-lactamase inhibitors, e.g., screening small molecule and peptide libraries for new β-lactamase inhibitors and penicillin-binding protein (PBP)-targeting antibiotics, and detecting β-lactamase-producing organisms in laboratory, environmental, or clinical sample matrices.

In one embodiment, the whole-cell biosensor assay comprises an E. coli cell transformed with a plasmid that includes a luxCDABE bioluminescent reporter controlled by an ampC promoter, such that exposure to a β-lactam antibiotic produces a luminescent signal. Results described below validate use of such a biosensor embodiment to measure β-lactamase activity and inhibition in whole cells. In addition, biosensor embodiments may also be used to measure β-lactamase activity and inhibition in cell lysates. Rather than monitoring changes in cell growth to measure β-lactamase inhibitor efficacy (as is done in conventional methods to determine minimum inhibitory concentrations (MICs)), which lacks specificity and may take up to 24 hours to provide a result, a biosensor assay according to embodiments described herein provides a more rapid and specific measure of inhibitor potency by measuring the ability of an inhibitor to rescue β-lactam antibiotics from β-lactamase-producing bacteria within a timeframe of only about 3.5 hours, with a high degree of specificity. The approach described herein may be extended to determine cellular IC50 values, allowing for inhibitor potency to be quantified.

In Gram-negative bacteria such as E. coli and various other pathogenic bacteria, β-lactamases are primarily located in the periplasm where they degrade β-lactams that have penetrated the outer cellular membrane. Therefore, β-lactamase inhibitors must also reach the periplasm to access their targets. This may account for differences between IC50 values for inhibitors against purified β-lactamases compared to IC50 values for inhibitors in a cellular assay. For example, several reported NDM-1 inhibitors (DPA, NTA, CAP) have IC50 values in the low micromolar range against purified enzymes, whereas, reported herein, these same inhibitors have IC50 values increased up to 4200-fold when evaluated against cells using the biosensor assay. This highlights the importance of evaluating the efficacy of β-lactamase inhibitors in a cellular context and demonstrates the utility of the whole-cell biosensor assay embodiments.

In other embodiments the biosensor may be used to detect β-lactamase (e.g., carbapenemase) producing organisms. As described in detail below, an embodiment of the biosensor assay was validated using laboratory E. coli strains producing an array of common carbapenemases (e.g., KPC-2, NDM-1, IMP-1, OXA-48), as well as a panel of clinical CPOs isolated at Kingston General Hospital (Kingston, Ontario, Canada). This method provided positive results within 2.5 hours inclusive of setup, with 100% sensitivity. A panel of clinical isolates collected from Shared Hospital Lab (Toronto, Ontario, Canada) that previously tested negative for carbapenemase production was used to assess the specificity of the assay; testing with this library using an assay according to one embodiment yielded a specificity of 90%, and using an assay according to another embodiment wherein proteinase K was added yielded a specificity of 100%.

As described in detail below, one embodiment successfully identified all OXA-48-producing isolates tested, addressing a key limitation that has been observed with the conventional CARBA-NP test. Thus, a biosensor assay as described herein may serve as an important tool in clinical microbiology labs for the rapid detection of CPOs, ensuring that clinicians can quickly administer or adjust antimicrobial therapy for patients suffering from potentially life-threatening infections caused by these pathogens, and that appropriate infection prevention control measures can be implemented when a CPO is detected.

In addition to providing a rapid test with high sensitivity and specificity, a biosensor assay as described herein exhibits a very low limit of detection (LOD). This may provide utility in critical applications such in prevention and control of outbreaks caused by CPOs. For example, in surveillance testing, which may be conducted via primary screening of patients who possess risk factors for CPO colonization (e.g., recent travel to certain geographical regions, past hospitalization or admission to the intensive care unit, previous carbapenem use, presence of central line devices, etc.), obtaining rapid and accurate results from primary samples (e.g., resuspended bacteria from rectal swabs) without the need for overnight culturing would be valuable in these situations. Due to the potentially high level of variability in CPO loads that occur in primary samples, a low LOD as provided by biosensor embodiments may help to accurately and rapidly detect the presence of CPOs in such samples.

Embodiments are further described by way of the following non-limiting Examples.

### Example 1

This example describes plasmid embodiments and their preparation.

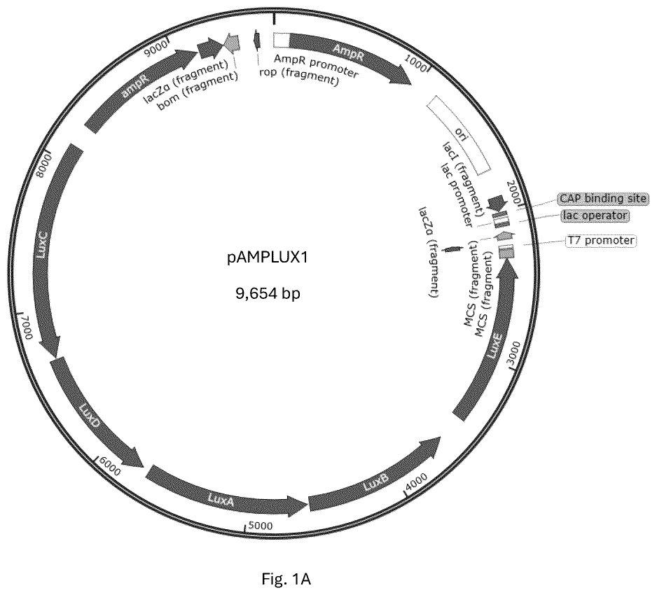

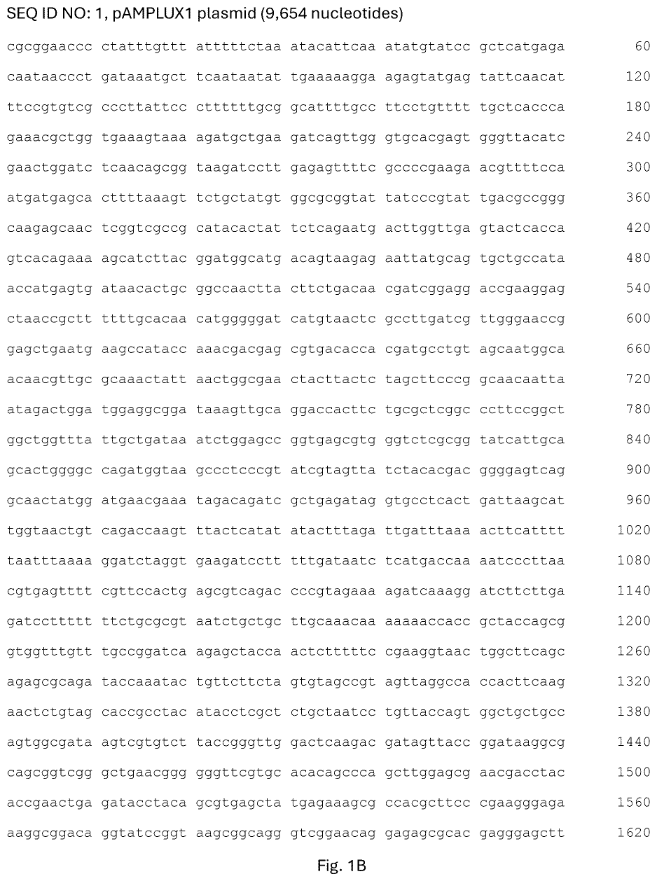

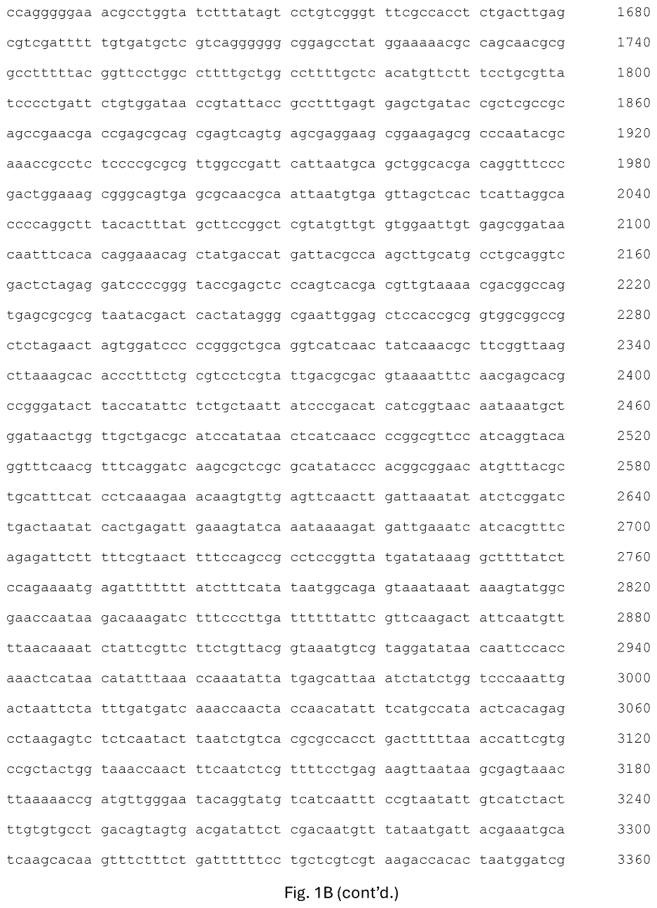

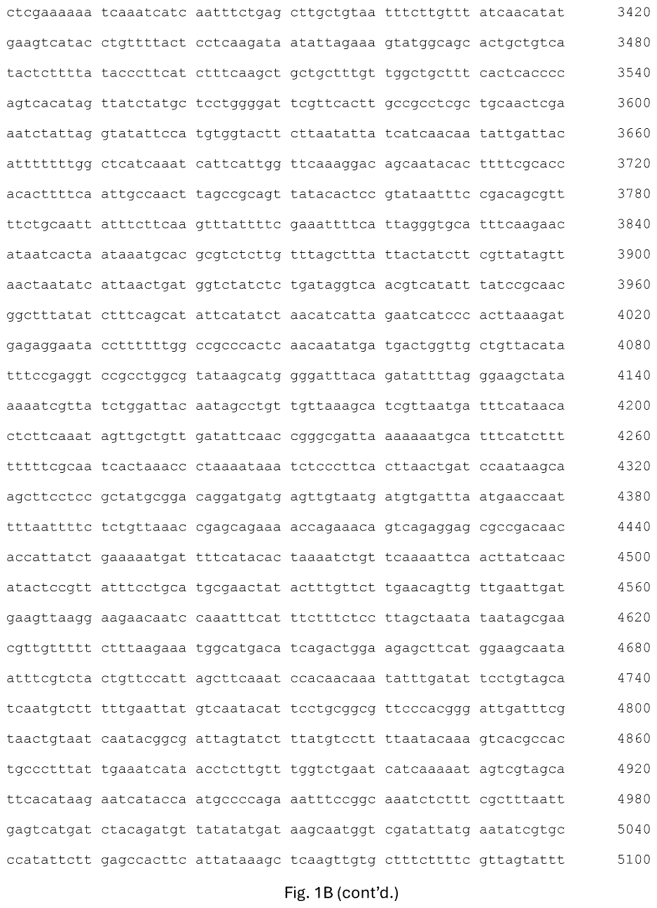

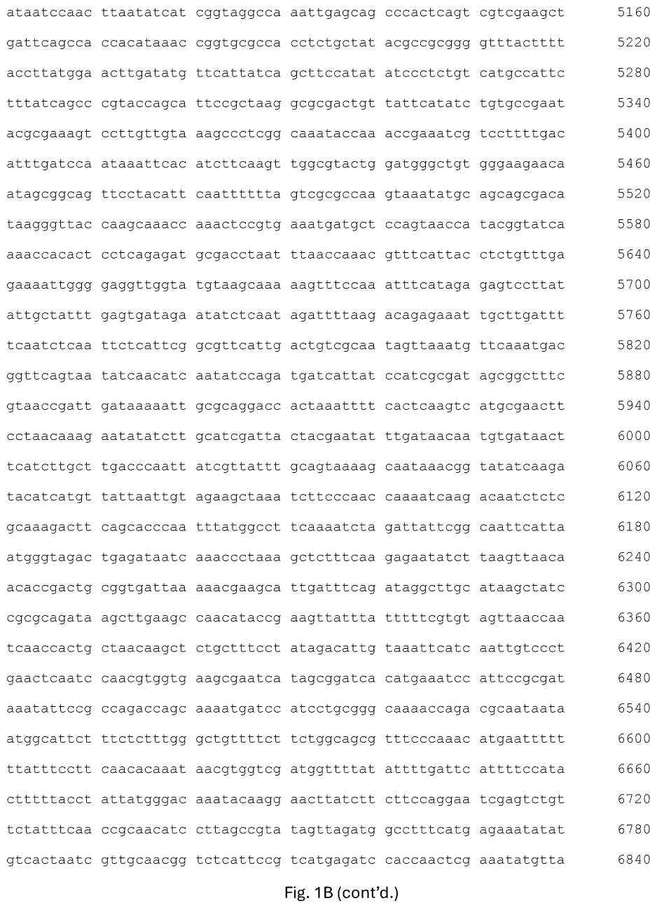

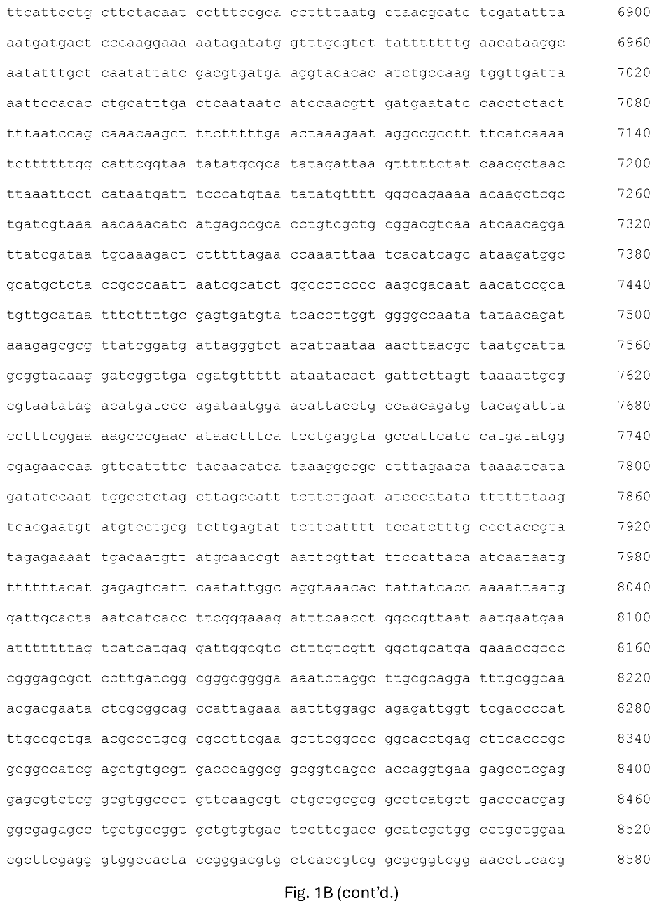

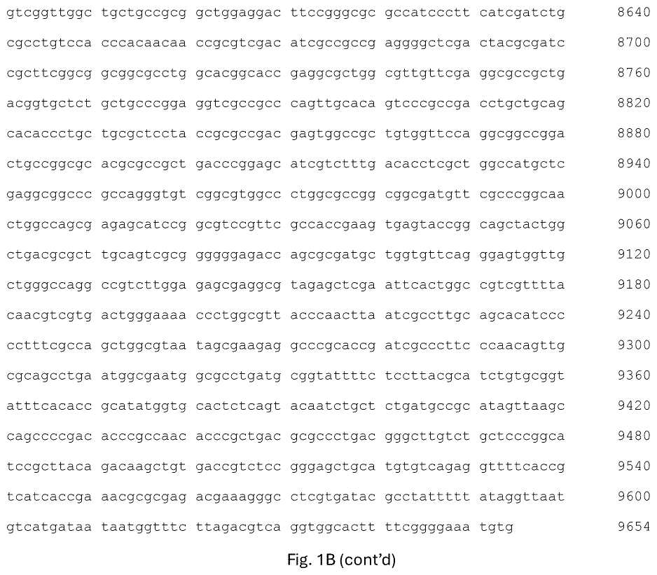

FIG. 1A is a map showing a plasmid construct according to one embodiment referred to herein as pAMPLUX1 (9,654 bp), and Table 1 lists nucleotide numbering for components of the plasmid. The complete DNA sequence is given in FIG. 1B and SEQ ID NO: 1.

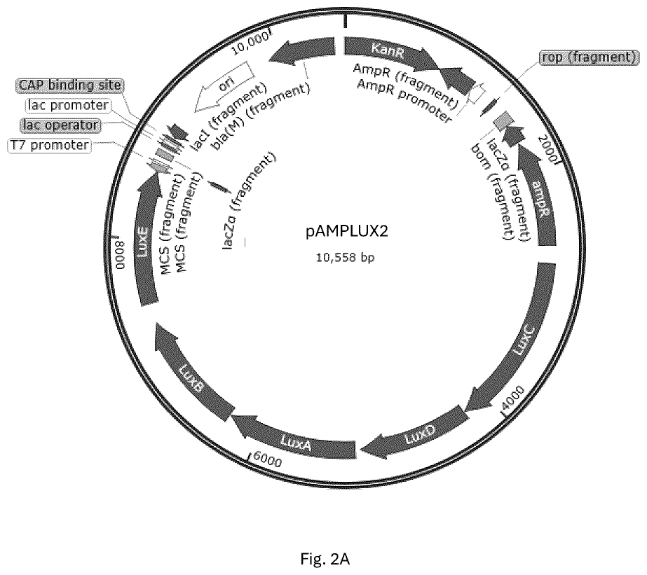

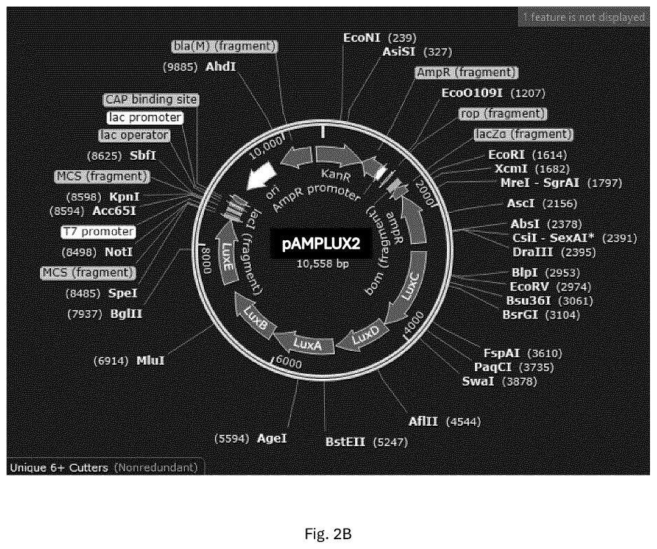

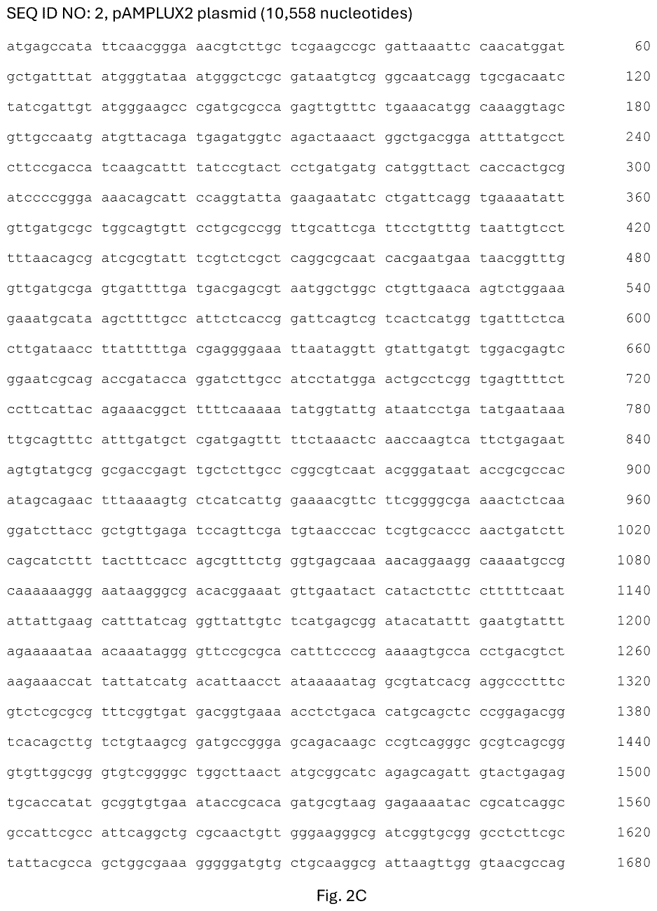

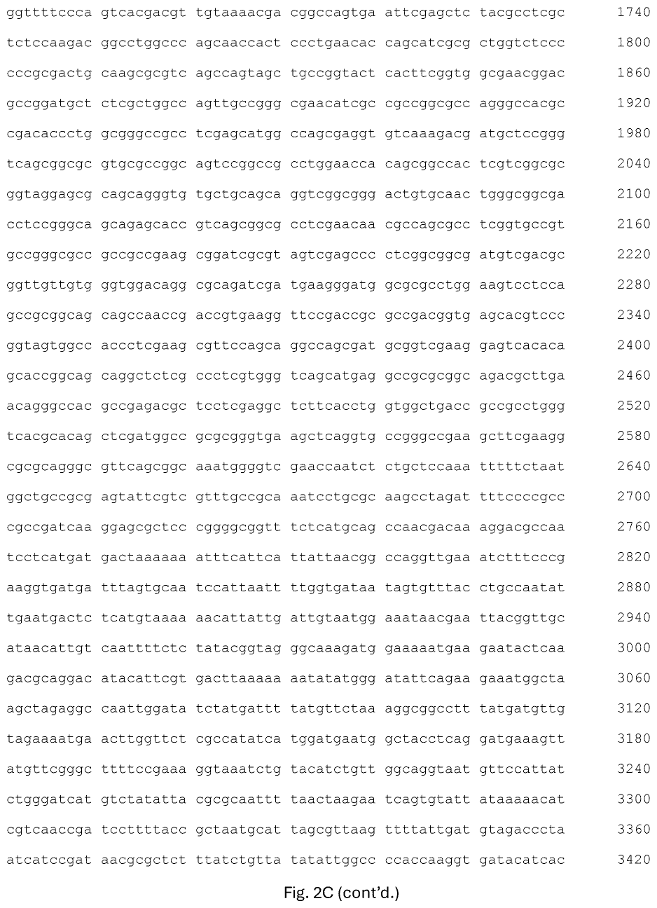

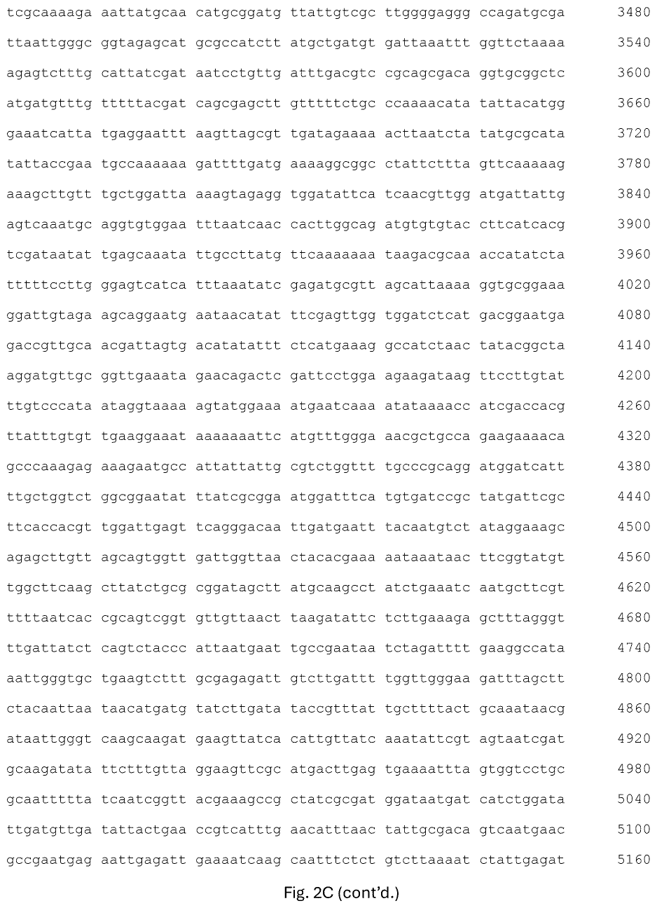

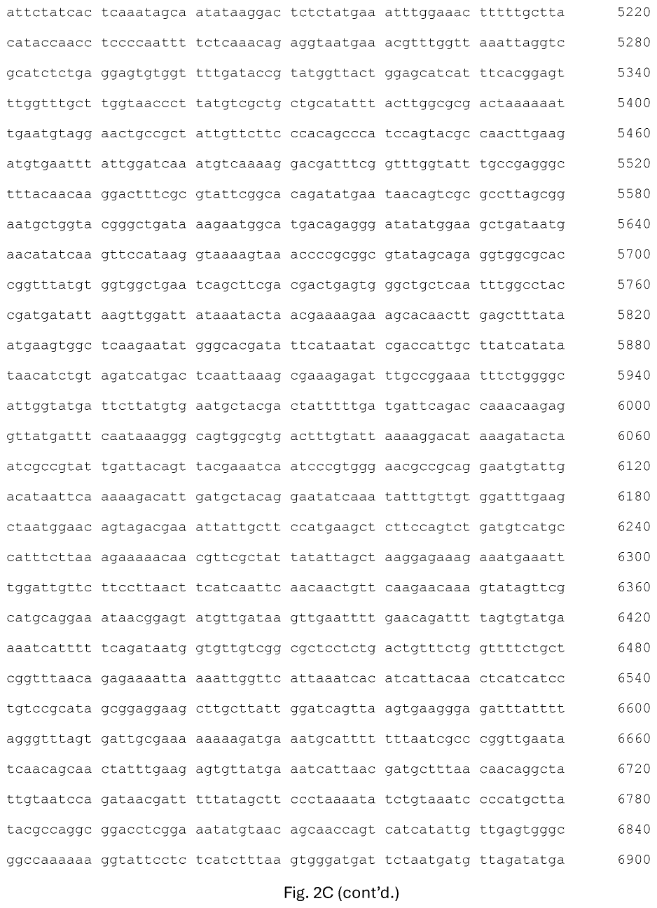

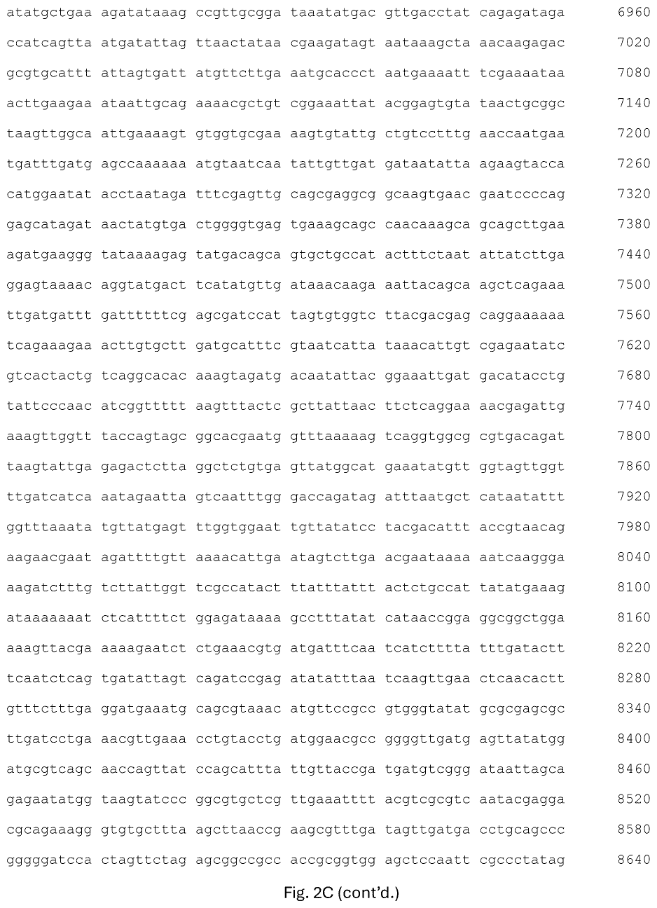

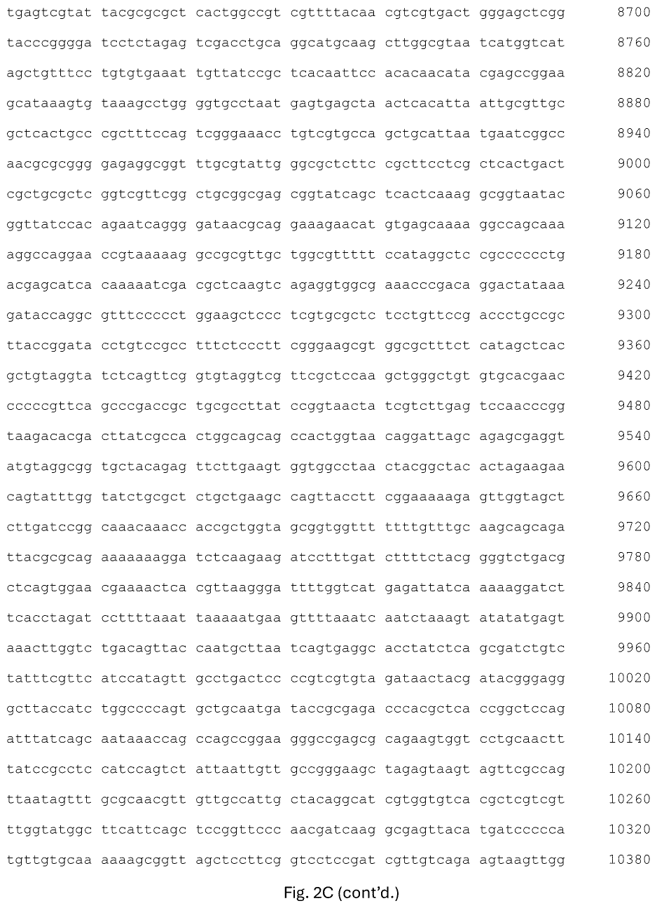

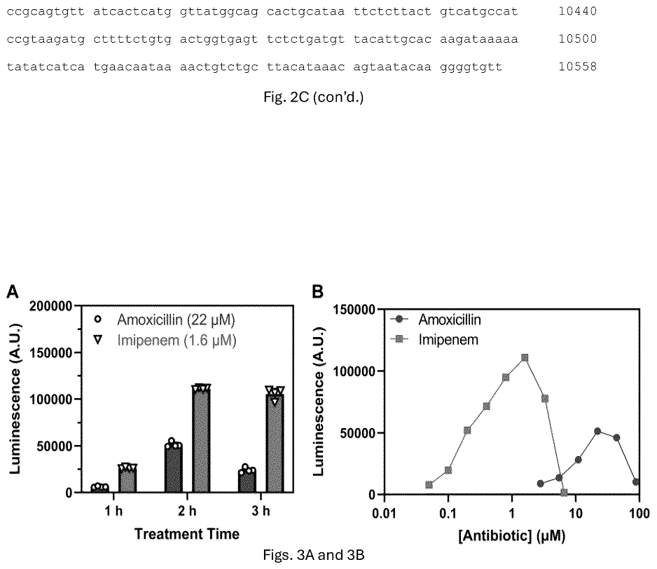

FIGS. 2A and 2B are maps showing plasmid constructs according to other embodiments referred to herein as pAMPLUX2 (10,558 bp), and Table 2 lists nucleotide numbering for components of the plasmid. The complete DNA sequence is given in FIG. 2C and SEQ ID NO: 2.

The plasmids encode the AmpR/ampC regulatory system from P. aeruginosa [18]. Exposure of P. aeruginosa to certain β-lactams elevates production of 1,6-anhydromuropeptides during peptidoglycan (PG) catabolismsome of these catabolites bind to AmpR inducing the expression of the ampC gene. The plasmids include the luxCDABE operon which includes five genes, producing a luciferase (luminescent protein encoded by luxA and luxB) and the biosynthetic enzymes (luxC, luxD), and luxE) that synthesize the substrate for the luciferase. In these plasmids, the luxCDABE operon is regulated by the ampC promoter such that exposure to a β-lactam antibiotic leads to the production of a luminescent signal. Upon degradation of the β-lactam antibiotic by β-lactamase prior to the addition of biosensor cells, no luminescence is produced. However, addition of an effective β-lactamase inhibitor protects the β-lactam, restoring luminescence.

To prepare the plasmids, the ampR gene and ampC promoter were amplified from the genomic DNA of P. aeruginosa 2_1_26 (BEI Resources) by polymerase chain reaction (PCR). The luxCDABE cassette was amplified from the pAKgfplux1 plasmid (Addgene #14083) by PCR. pUC19 vector (New England BioLabs) was digested with SacI (New England BioLabs) and purified by gel extraction following agarose gel electrophoresis. The PCR-amplified ampR/PampC and luxCDABE sequences were cloned into pUC19 by HiFi assembly (New England BioLabs) to generate the plasmid pAMPLUX1. To generate the plasmid pAMPLUX2, the ampicillin resistance marker of pAMPLUX1 was disrupted by restriction digest with Scal (New England Biolabs), and the PCR-amplified kanamycin resistance (kanR) gene from pHSG298 (National BioResource Project, NBRP) was cloned into this site using HiFi assembly. The sequences of pAMPLUX1 (SEQ ID NO: 1) and pAMPLUX2 (SEQ ID NO: 2) were confirmed by whole plasmid sequencing (Plasmidsaurus™ Inc.).

TABLE 1
Components of the pAMPLUX1 plasmid with nucleotide numbering
corresponding to the numbering in FIG. 1A and SEQ ID NO: 1.

| Biosensor Plasmid Component | Function of Component | Sequence Range on Plasmid |
|---|---|---|
| ampR promoter | Regulates expression of | 1→105 (105 bp) |
| | ampicillin resistance gene | |
| | (encodes for TEM-116 β- | |
| | lactamase) | |
| AmpR resistance enzyme | Encodes for TEM-116 β- | 106→966 (861 bp) |
| | lactamase, allowing for | |
| | selection of biosensor cells | |
| | in ampicillin-containing | |
| | media | |
| Intergenic region between | N/A | 967 → 1136 (170 bp) |
| AmpR and ori | | |
| ori | Origin of replication, | 1137→1725 (589 bp) |
| | allows for replication of the | |
| | plasmid in the bacteria | |
| Intergenic region between ori | N/A | 1726→1907 (182 bp) |
| and MCS fragment | | |
| Multiple Cloning Site fragment | N/A -genes are present in | 1908→2315 (408 bp) |
| (MCS), disrupted when cloning | the plasmid backbone, but | |
| in ampR/PampC + luxCDABE | are not being utilized for | |
| | the functionality of the | |
| | biosensor | |
| luxCDABE | Luminescent reporter under | 2316→8114 (5799 bp) |
| | the regulation of AmpR and | |
| | the ampC promoter | |
| ampC promoter/intergenic | Promoter which drives the | 8115 →8265 (151 bp) |
| region between ampR and | expression of the luxCDABE | |
| luxCDABE | cassette, which is activated | |
| | by carbapenem antibiotics | |
| ampR transcription factor | Transcription factor that | 8266 →9144 (879 bp) |
| | regulates | |
| | activation/repression of the | |
| | ampC promoter by | |
| | peptidoglycan fragments | |
| | produced during exposure | |
| | of biosensor cells to | |
| | carbapenem antibiotics | |
| Multiple Cloning Site fragment | N/A -genes are present in | 9145→9654 (510 bp) |
| (MCS), disrupted when cloning | the plasmid backbone, but | |
| in ampR/PampC + luxCDABE | are not being utilized for | |
| | the functionality of the | |
| | biosensor | |

TABLE 2
Components of the pAMPLUX2 plasmid with nucleotide numbering corresponding
to the numbering in FIG. 2A and 2B and SEQ ID NO: 2.

| Biosensor Plasmid Component | Function of Component | Sequence Range on Plasmid |
|---|---|---|
| Kanamycin resistance marker | Allows for selection of | 1 → 816 (816 bp) |
| (kanR) | biosensor cells in | |
| | kanamycin-containing | |
| | media | |
| AmpR (disrupted fragment) | N/A Fragment of | 817→1228 (412 bp) |
| | ampicillin-resistance gene, | |
| | which was disrupted (and | |
| | thus inactivated) by | |
| | cloning of the kanR gene | |
| Multiple Cloning Site fragment | N/A - consists of the | 1229→1738 (510 bp) |
| (MCS), disrupted when cloning | remainder of the MCS → | |
| in ampR/PampC + luxCDABE | these genes are present in | |
| | the plasmid backbone, but | |
| | are not being utilized for | |
| | the functionality of the | |
| | biosensor | |
| ampR transcription factor | Transcription factor that | 1739 →2617 (879 bp) |
| | regulates | |
| | activation/repression of | |
| | the ampC promoter by | |
| | peptidoglycan fragments | |
| | produced during exposure | |
| | of biosensor cells to | |
| | carbapenem antibiotics | |
| ampC promoter/intergenic | Promoter which drives the | 2618→ 2768 (151 bp) |
| region between ampR and | expression of the | |
| luxCDABE | luxCDABE cassette, which | |
| | is activated by | |
| | carbapenem antibiotics | |
| luxCDABE* | Luminescent reporter | 2769→ 8567 (5799 bp) |
| | under the regulation of | |
| | AmpR and the ampC | |
| | promoter | |
| Multiple Cloning Site fragment | N/A - consists of the | 8568 → 9157 (589 bp) |
| (MCS), disrupted when cloning | remainder of the MCS → | |
| in ampR/PampC + luxCDABE | these genes are present in | |
| | the plasmid backbone, but | |
| | are not being utilized for | |
| | the functionality of the | |
| | biosensor | |
| ori | Origin of replication, | 9158 →9746 (589 bp) |
| | allows for replication of | |
| | the plasmid in the bacteria | |
| Intergenic region between | N/A Fragment of | 9747 → 10558 (812 bp) |
| disrupted ori and disrupted | ampicillin-resistance gene, | |
| AmpR + AmpR (disrupted | which was disrupted (and | |
| fragment) | thus inactivated) by | |
| | cloning of the kanR gene | |

### Example 2

This example describes preparation and validation of a whole-cell luminescent biosensor embodiment.

E. coli BW25113 (NBRP) cells were transformed with the plasmid pAMPLUX of Example 1 to prepare a whole cell β-lactam-inducible biosensor. Exposure of biosensor cells to β-lactam antibiotics induces production of luminescence, which results from increased levels of 1,6-anhydromuropeptides produced as a result of β-lactam-induced peptidoglycan damage to the bacterial cell wall following inhibition of penicillin-binding protein 4 (PBP4). Upon degradation of the β-lactam by a β-lactamase, the luminescent signal of the biosensor cells is significantly reduced relative to the amount of signal produced during exposure of biosensor cells to β-lactam antibiotics. Thus, the biosensor cells may be used in an assay platform for, e.g., detecting and monitoring β-lactam degradation by β-lactamase-producing cells, assessing inhibitor activity, and rapidly detecting CPOs.

To validate the biosensor cells, treatment with imipenem and amoxicillin (β-lactams that induce the AmpR system) resulted in a strong luminescent signal (FIG. 3A) which was not observed for untransformed cells. Luminescence increased with antibiotic concentration until the amount of antibiotic was sufficient to kill the biosensor cells (FIG. 3B). For optimal sensitivity, subsequent β-lactamase inhibition studies used antibiotic concentrations that yielded the greatest biosensor induction. Additional optimization experiments were conducted to determine the optimal incubation temperature and culture density for subsequent assays. Biosensor cell density had no impact on the signal-to-background ratio under the conditions tested, while biosensor induction increased at higher temperatures.

### Example 3

This example describes methods for detecting and monitoring β-lactam degradation by β-lactamase-producing cells and assessing inhibitor activity using biosensor cells as described in Example 2.

The serine β-lactamase (SBL) TEM-116 is a penicillinase which hydrolyzes penicillins (e.g., amoxicillin (AMX)). After pre-treating TEM-116-producing E. coli cells with AMX, biosensor cells were added. After two hours of incubation, only low levels of luminescence were observed, indicating AMX degradation by TEM-116 (FIG. 3A). Pre-incubation of TEM-116 producing E. coli with AMX and SBL inhibitors tazobactam (TZB), clavulanic acid (CLAV) or avibactam (AVI) prior to the addition of biosensor cells resulted in an increase in luminescence, demonstrating the rescue of AMX (FIG. 4A).

The relationship between inhibitor concentration and biosensor induction in the presence of a fixed antibiotic concentration was then investigated (FIG. 4B). As the concentration of inhibitor was increased, the amount of luminescence also increased, indicating less antibiotic degradation by TEM-116. The resulting dose response curves were used to determine effective IC50 values for the inhibitors tested, quantifying their potencies in a cellular context. These data show that AVI is the most potent inhibitor of TEM-116, consistent with published reports for purified TEM-1 [19]. These results were verified with traditional turbidity-based assays by conducting growth-based experiments under the same conditions as the biosensor assays. It should be noted that TZB, AVI, and CLAV induce anhydromuropeptide production and can activate the biosensor in the absence of antibiotic. However, the levels of induction were relatively low at the concentrations tested when compared to their antibiotic partners, and were accounted for during inhibition studies. The potential inhibition of penicillin-binding proteins (PBPs) by new β-lactamase inhibitors can be assessed by testing the inhibitors against biosensor cells in the absence of β-lactams and β-lactamase-producing cells.

Following the experiments with TEM-116, inhibition studies were extended to include KPC-2 and OXA-48, SBLs of clinical significance due in part to their ability to degrade carbapenem antibiotics. While treatment of E. coli cells that produce KPC-2 or OXA-48 with AVI rescued AMX, leading to a strong luminescent signal, TZB and CLAV did not inhibit these enzymes under the conditions tested (FIGS. 4C and 4D). Dose response analyses indicated that avibactam inhibited KPC-2 and OXA-48 with similar potencies (FIG. 4E). Notably, while much of the inhibition data is in agreement with published values, some inhibitor potencies were moderately different from reported values obtained with purified enzymes [20-22]. These differences likely result from the choice of β-lactam used for the inhibition studies, as amoxicillin was used herein while most other studies employed chromogenic or fluorogenic cephalosporin derivatives. These differences may also reflect the impact of cellular factors on inhibitor potency, including the level of β-lactamase production and cell wall permeability.

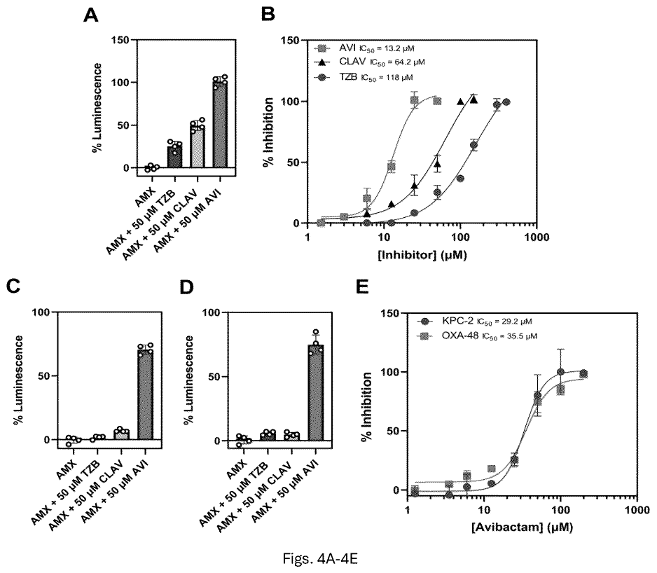

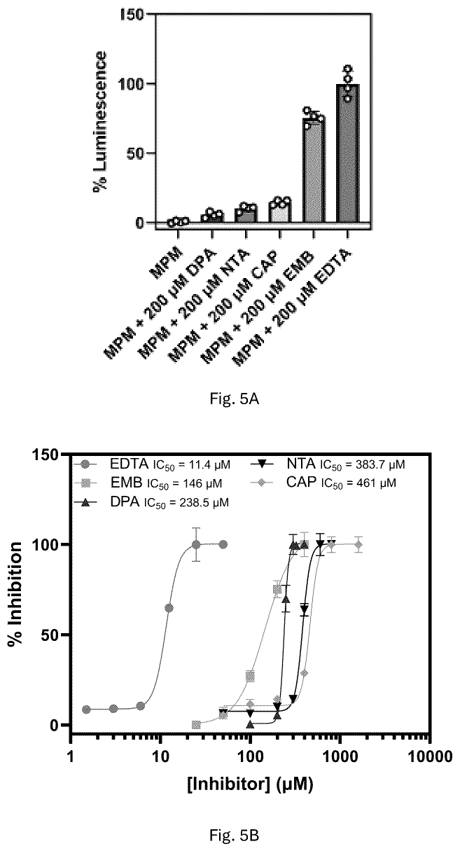

Inhibition studies were expanded to the MBLs NDM-1, IMP-1, and VIM-2. As there are not currently any MBL inhibitors approved for clinical use, five inhibitors described in the literature were tested: ethylenediaminetetraacetic acid (EDTA), dipicolinic acid (DPA), nitrilotriacetic acid (NTA), captopril (CAP), and embelin (EMB) [23-25]. Assay results suggest that EDTA is the most potent inhibitor against NDM-1 (FIGS. 5A and 5B), followed by EMB, DPA, NTA, and finally CAP. These trends are consistent with published IC50 values for these inhibitors against purified NDM-1 [23-26]. While EDTA was also the most effective inhibitor against IMP-1 and VIM-2, EMB and NTA exhibited poor activity against IMP-1 in agreement with literature reports [24, 25].

It should be noted that the cellular IC50 values obtained in this study may be impacted by several factors. Firstly, the inoculum effect (i.e., an increase in the MIC of an antimicrobial agent when the inoculum size of test bacteria is increased) should be considered. We found that the inoculum of β-lactamase-producing cells can be adjusted to identify inhibitors of higher or lower potency. Cellular IC50 values could also be impacted by the destabilization of the outer membrane by metal chelating inhibitors (e.g., EDTA), which allows easier passage of small molecules across the cell membrane. This could increase the entry of β-lactams and β-lactamase inhibitors, resulting in increased potency of these agents. β-Lactamase inhibitors that also permeabilize the outer membrane (e.g., EDTA) can be identified by assessing luminescence production following treatment of biosensor cells with inhibitor alone, or in combination with β-lactams in the absence of a β-lactamase.

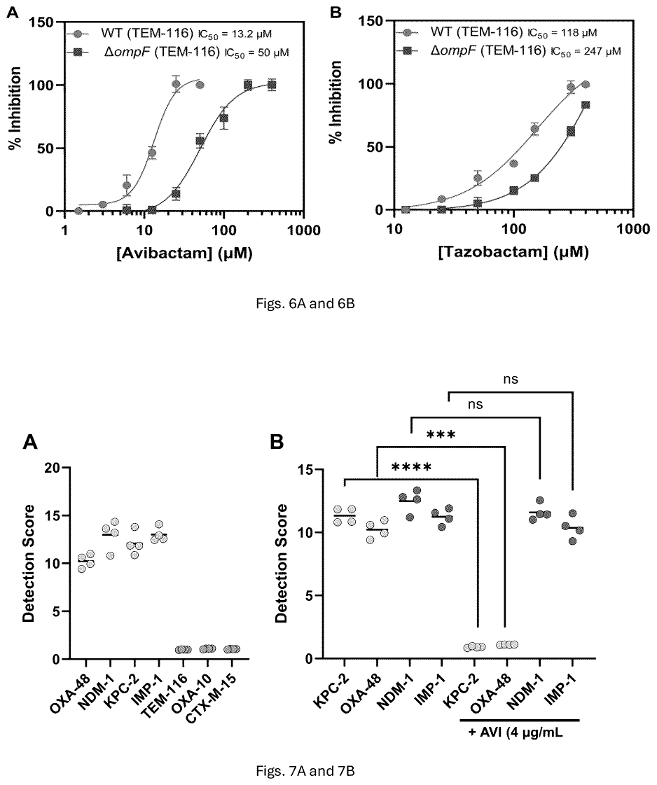

Beyond the production of β-lactamases, Gram-negative pathogens employ other resistance mechanisms that impact the efficacy of β-lactams and β-lactamase inhibitors (e.g., reduced porin expression, efflux pumps). While purified enzyme assays cannot account for the presence of other resistance mechanisms, the biosensor can evaluate the impact of cellular factors such as decreased permeability. The E. coli porin OmpF is used by certain β-lactams for cellular entry, and decreased production of this porin is associated with resistance to β-lactams and β-lactam-β-lactamase inhibitor combinations (e.g., ceftazidime-avibactam). The biosensor was used to evaluate the efficacy of β-lactamase inhibitors against an E. coli AompF strain that produces TEM-116. A notable increase in IC50 was observed for both AVI and TZB when administered with AMX against this strain (FIGS. 6A and 6B), highlighting the importance of membrane permeability on the efficacy of β-lactam-β-lactamase inhibitor combinations. Based on these results, inhibitor discovery campaigns may use the biosensor to simultaneously screen compound libraries against a panel of strains encoding representative β-lactamases in combination with other resistance mechanisms.

### Example 4

This example describes methods for detecting carbapenemase-producing organisms using the luminescent biosensor of Example 2, referred to as biosensor cells. Two embodiments of an assay were tested, one without proteinase K and one with proteinase K added after an incubation for imipenem hydrolysis.

### Reagents

BD BBL cation-adjusted Mueller-Hinton Broth (CAMHB) and proteinase K were purchased from Fisher Scientific. Imipenem was purchased from Glentham Life Sciences. Avibactam was purchased from Medkoo Biosciences. BugBuster protein extraction reagent was purchased from Millipore Sigma. Nitrocefin was purchased from TOKU-E.

### Bacterial Strains and Clinical Isolates

E. coli BW25113 cells were transformed with pACYC184 vectors encoding representative penicillinases, extended spectrum β-lactamases (ESBLs), and carbapenemases. These laboratory strains are listed in Table 3. All CPO clinical isolates used in this study were collected by the microbiology labs at Kingston General Hospital (Kingston, Ontario, Canada) and Sunnybrook Hospital (Toronto, Ontario, Canada) during the normal course of care, and included 50 CPOs belonging to several bacterial species (see Table 4). The CPOs had been previously tested by the microbiology lab for carbapenemase production via the mCIM test, and the presence and identity of carbapenemase genes was tested by PCR. Non-CPO clinical isolates were collected at Shared Hospital Laboratory (Toronto, Ontario, Canada), and included ESBL-producing E. coli, Klebsiella and Proteus strains (see Table 5).

### Minimum Inhibitory Concentration (MIC) Testing

All MIC testing was conducted according to Clinical and Laboratory Standards Institute (CLSI) guidelines [27]. Test isolates were cultured for 16-20 h on CAMHB agar plates (supplemented with the appropriate antibiotic where necessary for lab CPO strains). Imipenem stocks were prepared in sterile water and serially diluted in CAMHB in clear sterile 96-well non-treated flat-bottom plates (Falcon). Cell suspensions were prepared by resuspending colonies in CAMHB to OD₆₀₀=0.1 (approx. 1×10⁸ CFU/mL). These suspensions were diluted in CAMHB to approx. 5×10⁶ CFU/mL, and 20 μL of the diluted suspensions was added to 180 μL of each of the imipenem dilutions. Plates were incubated for 20 h at 37° C. without shaking and OD₆₀₀ readings were collected using a Synergy LX plate reader (Agilent BioTek) to determine MICs.

### Laboratory Strain Carbapenemase Detection Assay

Laboratory strains (Table 3), a carbapenemase-negative control (E. coli BW25113) and biosensor cells were cultured for 16-20 h on CAMHB agar (supplemented with the appropriate antibiotic where necessary). Imipenem stocks were prepared in sterile water and diluted in the assay medium (CAMHB) to a final concentration of 1 μg/mL.

### i) Colony Resuspension Method

A biosensor cell suspension was prepared by suspending biosensor cell colonies in CAMHB to an OD₆₀₀=0.10-0.12, approximately 0.5 McFarland units (MFU). For direct colony assays, a 1 μL loopful of test cells was transferred to a 5 mL solution of imipenem (1 μg/mL) in CAMHB. After mixing, 150 μL of this test solution was immediately transferred to 50 μL of the biosensor cell suspension in the wells of a white opaque 96-well plate (Lumitrac 200, Greiner Bio-One). Test mixtures were incubated at 37° C. without shaking for 90 minutes, and luminescence readings were taken using a Synergy LX multimode plate reader (Agilent BioTek). Detection scores were determined by dividing the luminescent readings for a carbapenemase-negative sample by the luminescent readings for the test samples.

### ii) Limit of Detection Testing in Liquid Samples

Wild-type E. coli BW25113 and E. coli BW25113 transformed with pACYC184 vectors encoding NDM-1, IMP-1, OXA-48, or KPC-2 (24) were used to inoculate 5 mL CAMHB (supplemented with 25 μg/mL chloramphenicol for E. coli transformed with pACYC184 vectors). These cultures were grown overnight at 37° C., 200 rpm. The following day, the cultures were diluted to an OD600=0.1 (corresponding to an approximate CFU/mL of 1×10⁸). To test the limit of detection of the assay, further ten-fold serial dilutions were prepared in CAMHB to approximate CFU/mL values ranging from 1×10⁷ to 1×10². Imipenem was added to a final concentration of 1 μg/mL to each tube, and the mixtures were then incubated at 37° C., 200 rpm for 4 h. After incubation, these cultures were centrifuged (13,000 rpm) and 150 μL of the supernatants were transferred to 50 μL aliquots of biosensor cell suspensions (approx. 0.5 MFU) in the wells of a white opaque 96-well plate (Lumitrac 200, Greiner Bio-One). The plate was incubated at 37° C. without shaking for 90 minutes, and luminescence readings were taken using a Synergy LX multimode plate reader (Agilent BioTek). All strains used in the colony and liquid sample assays were tested in quadruplicate.

### Cell Lysis Testing

E. coli BW25113 cells transformed with pACYC184-NDM-1 were cultured for 16-20 h at 37° C. on CAMHB agar plates supplemented with 25 μg/mL chloramphenicol. The following day, serial dilutions of BugBuster protein extraction reagent were prepared in autoclaved water to 100, 50, 25, 10 and 0% v/v. Then 50 μL of each dilution was transferred to 1.5 mL Eppendorf tubes. A 1 μL loopful of NDM-1-producing E. coli was transferred to these lysis solutions, which were then incubated at room temperature for 10 min. Cellular debris was pelleted for 3 min using a Fisherbrand 100-240V Mini-Centrifuge (fixed speed of 6,000 rpm). Following centrifugation, 10 μL of each supernatant was diluted in 990 μL of CAMHB, and 90 μL of the resulting solution was added to the wells of a clear 96-well plate (Falcon). Next, 10 μL of a 2 mM nitrocefin stock was added and reactions were monitored at 488 nm for 10 min using a Synergy LX multimode plate reader (Agilent BioTek).

### Carbapenemase Detection in Clinical Isolates

Clinical isolates (Tables 4 and 5), a carbapenemase-negative control (E. coli ATCC 25922), and biosensor cells were cultured for 16-20 h on CAMHB agar at 37° C. Imipenem stocks were prepared in sterile water. Proteinase K stocks were prepared to 20 mg/mL in sterile water and aliquots were stored at −20° C. until use. Two 1 μL loopful of test cells was added to 50 μL of 40% v/v BugBuster solution and incubated at room temperature for 10 min. Cellular debris was pelleted for 3 min using a Fisherbrand 100-240V Mini-Centrifuge (fixed speed of 6,000 rpm). Following lysis, 10 μL of lysate was added to 990 μL of CAMHB containing 1 μg/mL imipenem, and the mixture was incubated at 37° C. to allow for imipenem hydrolysis to occur. In one embodiment, following this incubation, proteinase K was added to each tube to a final concentration of 100 μg/mL. Next, 150 μL of the resulting solution was mixed with 50 μL of a 0.5 MFU suspension of biosensor cells in the wells of a white opaque 96-well plate (Lumitrac 200, Greiner Bio-One). These test mixtures were incubated at 37° C. without shaking for 90 min, and luminescence readings were taken using a Synergy LX multimode plate reader (Agilent BioTek). Detection scores were determined by dividing the luminescent readings for a carbapenemase-negative sample by the luminescent readings for the test samples. All isolates were tested in triplicate.

### Statistical Analysis

All statistical tests were conducted using GraphPad Prism, version 10.1. In all cases, un-paired t-tests with Welch's correction were used to determine statistical significance between detection scores for relevant treatment populations.

### Results

Testing of the biosensor performance with E. coli BW25113 strains producing some of the most prevalent plasmid-encoded carbapenemases (i.e., KPC-2, NDM-1, IMP-1, and OXA-48) yielded high detection scores (FIG. 7A). These strains exhibited varying levels of susceptibility to the carbapenem imipenem (Table 3). Detection scores were determined by dividing the strong luminescent readings for a carbapenemase-negative sample by the luminescent readings for the test samples. Notably, the strain producing the OXA-48 carbapenemase tested strongly positive, with detection scores comparable to the other CPOs. OXA-48 enzymes are difficult to detect due to their relatively weak hydrolytic activity against carbapenems, compared to other types of carbapenemases. This confirms the utility of the biosensor for detecting OXA-48-type enzymes, an important result as these enzymes have increased greatly in global prevalence. In addition to testing the wild-type carbapenemase-producing E. coli strains, a series of strains lacking OmpF, a key porin used for imipenem entry, were also tested. Although the ΔompF strains gave slightly reduced detection scores compared to wild-type strains, all samples still tested strongly positive.

TABLE 3
Imipenem susceptibility of E. coli laboratory strains.

| | Imipenem | | |
|---|---|---|---|
| | MIC | Detection | Biosensor |
| E. coli strain | (μg/mL) | Score | Result |
| ATCC 25922 | 0.25 | 1 | Negative |
| BW25113 | 0.25 | 1 | Negative |
| BW25113 ΔompF | 0.50 | 1 | Negative |
| BW25113 + pACYC184-NDM-1 | 512 | 12.9 | Positive |
| BW25113 + pACYC184-IMP-1 | 256 | 12.9 | Positive |
| BW25113 + pACYC184-KPC-2 | 256 | 12.1 | Positive |
| BW25113 + pACYC184-OXA-48 | 4 | 10.2 | Positive |
| BW25113 + pACYC184-CTX-M-15 | 0.25 | 1.03 | Negative |
| BW25113 + pACYC184-OXA-10 | 0.25 | 1.07 | Negative |
| BW25113 + pACYC184-TEM-116 | 0.25 | 0.98 | Negative |

E. coli laboratory strains expressing plasmid-encoded penicillinases and ESBLs (i.e., TEM-116, CTX-M-15, OXA-10) were used to assess the specificity of the assay as these β-lactamases cannot efficiently degrade imipenem. The penicillinase- and ESBL-producing strains yielded low detection scores comparable to that obtained for a β-lactamase-negative control strain (FIG. 7A).

To investigate the use of the biosensor assay to evaluate the efficacy of β-lactamase inhibitors against the carbapenemase produced by a CPO, a second treatment was carried out wherein a fixed concentration of the β-lactamase inhibitor avibactam (4 μg/mL) in combination with imipenem was used, and the detection scores were compared to samples treated with imipenem alone (FIG. 7B). KPC-2- and OXA-48-producing strains demonstrated notable reductions in detection scores following the combined imipenem+avibactam treatment, compared to imipenem alone. In contrast, NDM-1- and IMP-1-producing strains did not exhibit a significant reduction in score, indicating avibactam did not prevent the hydrolysis of imipenem by these CPOs. These results are consistent with the inhibitory mechanism of avibactam, which targets serine β-lactamases (like KPC-2 and OXA-48) but not metallo-β-lactamases (like NDM-1 and IMP-1).

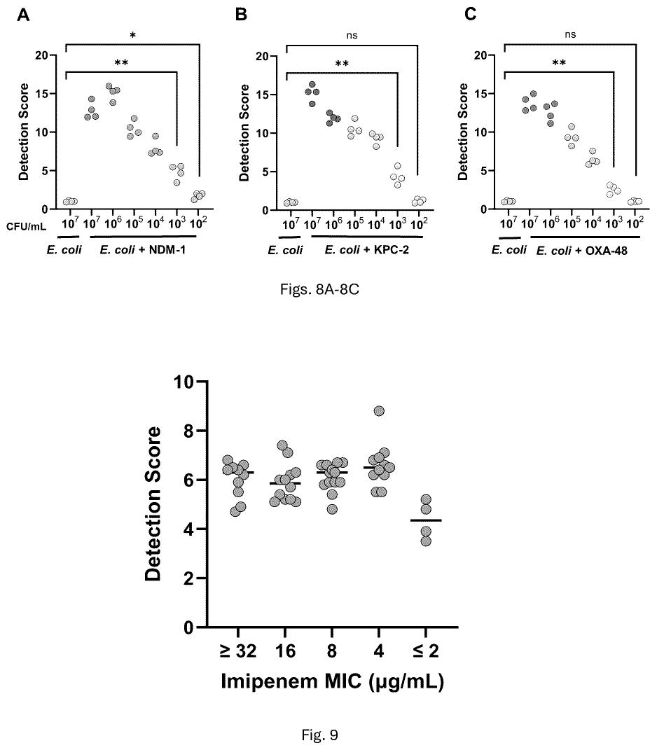

To determine the limit of detection (LOD) of the biosensor, tubes of CAMHB supplemented with 1 μg/mL imipenem were spiked with NDM-1-, KPC-2- or OXA-48-producing E. coli lab strains to final CFU/mL dilutions ranging from approximately 1×10⁷ to 1×10² CFU/mL. The tubes were incubated for 4 hours at 37° C. to allow for bacterial growth and imipenem degradation to occur before mixing the contents with biosensor cells. Using this approach, the biosensor was able to detect the NDM-1-producing strain at very low levels down to 10² CFU/mL and the KPC-2- and OXA-48-producing strains down to 10³ CFU/mL (FIGS. 8A-8C, respectively).

The biosensor assay was also evaluated for efficacy in detecting clinical CPO isolates. However, the detection scores observed for several of the clinical strains were significantly lower than those obtained with the laboratory CPO strains. Some potential factors that could have impacted carbapenemase detection include reduced outer membrane permeability of imipenem or lower levels of carbapenemase production relative to the previously tested lab strains.

To address the potential impact of cell wall permeability on detection, an additional step was introduced in which test strains were lysed using BugBuster protein extraction reagent. Prior to use in clinical testing, the concentration of lysis solution needed to be optimized such that the test strain was successfully lysed with minimal downstream impact on the biosensor cells. Cell lysis of E. coli BW25113 cells was effective at concentrations of BugBuster greater than 25% v/v. BugBuster solutions of 50% and 100% v/v had significant impacts on luminescence production by the biosensor cells, while the 25% solution did not. Thus, a solution containing 30% v/v BugBuster was used to balance lysis effectiveness while minimizing the impact on biosensor cells.

As low carbapenemase expression or activity could impact detection, a pre-incubation step was introduced in which test lysates were incubated with imipenem for 30 min prior to the addition of biosensor cells. Several clinical isolates producing OXA-48, a carbapenemase exhibiting weak hydrolytic activity against carbapenems, were tested, comparing detection scores with and without the 30 min pre-incubation step. Pre-incubating cell lysates with imipenem prior to the addition of biosensor cells resulted in markedly increased detection scores for isolates that tested weakly positive without pre-incubation.

Once the final workflow of the assay was established, the sensitivity of the biosensor assay was tested using a panel of 50 clinical CPO isolates. The overall sensitivity of the assay was 100%, with all NDM-(n=12/12), VIM-(n=14/14), KPC-(n=15/15), KPC+NDM-(n=1/1) and OXA-48-(n=8/8) producing isolates testing positive (Table 4). Biosensor scores remained consistent across groups of isolates with MICs≥4 μg/mL, with average detection scores greater than 5.9 (FIG. 9). A reduction in the average detection score to 4.3 was only observed for isolates with low imipenem MICs (≤2 μg/mL). For the embodiment wherein proteinase K was not added after the imipenem hydrolysis incubation, the overall specificity of the assay was 90%, with 27/30 non-CPO isolates testing negative (Table 5). Further testing suggested the three E. coli isolates which tested false positive may be producing a biomolecule that is negatively impacting luminescence production by the biosensor cells. Accordingly, to address this another embodiment included adding proteinase K after the imipenem hydrolysis incubation. In this embodiment the overall specificity of the assay was 100%, with 30/30 non-CPO isolates testing negative (Table 5—see Isolate Nos. 4, 23, 24).

TABLE 4
Imipenem susceptibility, detection scores, and biosensor test
results for carbapenemase-producing clinical isolates.

| | | | Imipenem | | |
|---|---|---|---|---|---|
| Isolate | | | MIC | Detection | Biosensor |
| No. | Species | Enzyme | (μg/mL) | Score | Result |
| 1 | C. freundii | VIM | 8 | 5.8 | Positive |
| 2 | C. freundii | VIM | 16 | 5.7 | Positive |
| 3 | C. freundii | KPC | 8 | 6.6 | Positive |
| 4 | C. freundii | KPC | 4 | 6.6 | Positive |
| 5 | C. freundii | KPC | 8 | 6.3 | Positive |
| 6 | C. freundii | KPC | 8 | 6.7 | Positive |
| 7 | C. freundii | KPC + | 16 | 6.2 | Positive |
| | | NDM | | | |
| 8 | E. coli | VIM | 8 | 6.6 | Positive |
| 9 | E. coli | NDM | 16 | 6 | Positive |
| 10 | E. coli | NDM | 8 | 5.9 | Positive |
| 11 | E. coli | NDM | 16 | 5.2 | Positive |
| 12 | E. coli | NDM | >32 | 5.5 | Positive |
| 13 | E. coli | NDM | 8 | 6.7 | Positive |
| 14 | E. coli | NDM | 8 | 6.4 | Positive |
| 15 | E. coli | NDM | >32 | 6.6 | Positive |
| 16 | E. coli | KPC | 4 | 6.2 | Positive |
| 17 | E. coli | KPC | 2 | 4.8 | Positive |
| 18 | E. coli | KPC | 4 | 5.5 | Positive |
| 19 | E. coli | KPC | 4 | 6.5 | Positive |
| 20 | E. coli | KPC | 8 | 5.4 | Positive |
| 21 | E. coli | OXA-48 | 2 | 3.5 | Positive |
| 22 | E. coli | OXA-48 | 2 | 3.9 | Positive |
| 23 | E. coli | OXA-48 | 32 | 6.2 | Positive |
| 24 | E. coli | OXA-48 | 16 | 5.1 | Positive |
| 25 | E. coli | OXA-48 | 16 | 5.4 | Positive |
| 26 | E. coli | OXA-48 | 2 | 5.2 | Positive |
| 27 | E. coli | OXA-48 | 16 | 5.1 | Positive |
| 28 | E. coli | OXA-48 | 32 | 4.7 | Positive |
| 29 | E. cloacae | VIM | 32 | 4.9 | Positive |
| 30 | E. cloacae | VIM | >32 | 6.4 | Positive |
| 31 | E. cloacae | VIM | 16 | 5.2 | Positive |
| 32 | E. cloacae | VIM | 32 | 5.9 | Positive |
| 33 | E. cloacae | VIM | 16 | 6 | Positive |
| 34 | E. cloacae | VIM | 4 | 6.2 | Positive |
| 35 | E. cloacae | VIM | 4 | 6.4 | Positive |
| 36 | E. cloacae | VIM | 16 | 7.1 | Positive |
| 37 | E. cloacae | VIM | 16 | 7.44 | Positive |
| 38 | E. cloacae | NDM | 8 | 4.8 | Positive |
| 39 | E. cloacae | NDM | 16 | 6.3 | Positive |
| 40 | E. cloacae | NDM | >32 | 6.8 | Positive |
| 41 | K. oxytoca | KPC | 32 | 6.5 | Positive |
| 42 | K. pneumoniae | VIM | 4 | 8.8 | Positive |
| 43 | K. pneumoniae | NDM | 32 | 6.4 | Positive |
| 44 | K. pneumoniae | NDM | 4 | 5.5 | Positive |
| 45 | K. pneumoniae | KPC | 8 | 6.3 | Positive |
| 46 | K. pneumoniae | KPC | 4 | 7.1 | Positive |
| 47 | K. pneumoniae | KPC | 4 | 6.8 | Positive |
| 48 | K. pneumoniae | KPC | 8 | 5.9 | Positive |
| 49 | K. pneumoniae | KPC | 4 | 6.9 | Positive |
| 50 | M. morganii | VIM | 8 | 5.9 | Positive |

TABLE 5
Biosensor test results for non-CPO
clinical isolates producing ESBLs.

| Isolate No. | Species (Class of ESBL) | Biosensor Result |
|---|---|---|
| 1 | E. coli (Class D) | Negative |
| 2 | E. coli (Class C) | Negative |
| 3 | K. pneumoniae (Class A) | Negative |
| 4 | E. coli (Class C) | Positive |
| | | Negative with |
| | | proteinase K |
| 5 | K. oxytoca (Class A) | Negative |
| 6 | K. oxytoca (Class A) | Negative |
| 7 | K. pneumoniae (Class A) | Negative |
| 8 | E. coli (Class A) | Negative |
| 9 | E. coli (Class A) | Negative |
| 10 | E. coli (Class A + C) | Negative |
| 11 | K. pneumoniae (Class C) | Negative |
| 12 | E. coli (Class A + C) | Negative |
| 13 | E. coli (Class A + C) | Negative |
| 14 | K. pneumoniae (Class A + C) | Negative |
| 15 | K. pneumoniae (Class A + C) | Negative |
| 16 | P. mirabilis (Class A) | Negative |
| 17 | P. mirabilis (Class A) | Negative |
| 18 | K. oxytoca (Class A) | Negative |
| 19 | K. oxytoca (Class D) | Negative |
| 20 | P. mirabilis (Class A) | Negative |
| 21 | E. coli (Class C) | Negative |
| 22 | E. coli (Class C) | Negative |
| 23 | E. coli (Class C) | Positive |
| | | Negative with |
| | | proteinase K |
| 24 | E. coli (Class C) | Positive |
| | | Negative with |
| | | proteinase K |
| 25 | K. pneumoniae (Class A) | Negative |
| 26 | K. pneumoniae (Class A) | Negative |
| 27 | K. pneumoniae (Class A) | Negative |
| 28 | K. oxytoca (Class A) | Negative |
| 29 | K. oxytoca (Class A) | Negative |
| 30 | K. oxytoca (Class A) | Negative |

Overall, the sensitivity (100%) and specificity (100%) of the biosensor assay according to this example are comparable to currently used methods for CPO detection, while also addressing drawbacks associated with such methods. The sensitivity of the mCIM test has been reported as ranging from 90.4 to 100%, demonstrating it is a robust method for detecting CPOs. In a multicenter evaluation of the mCIM assay conducted by Simner et al., the mCIM was very effective in detecting carbapenemase-producing P. aeruginosa (sensitivity and specificity of 98 and 95% respectively) [28]. A key drawback of the mCIM assay is the relatively long turnaround time; as the primary readout of the mCIM is cell growth, samples must be incubated for 16-20 h prior to interpretation. In contrast, a biosensor assay as described herein provides a significant improvement, providing results in just 2.5 h.

The currently employed CARBA-NP test provides positive test results in approx. 2 hours. This test exhibits a high degree of sensitivity for the detection of KPC, NDM, IMP and VIM-type enzymes. However, several reports indicate the CARBA-NP test exhibits lower sensitivity for isolates producing carbapenemases with weaker hydrolytic activities [29, 30]. Given these limitations, the efficacy of the CARBA-NP test was compared to the biosensor assay embodiment for the detection of OXA-48-producing isolates in the above CPO library. Overall, the CARBA-NP yielded positive results for 6/8 OXA-48-producing E. coli isolates, while the biosensor assay successfully detected all 8/8 OXA-48-producers. Although only a small library of OXA-48-producing isolates was tested in this study, it is expected that embodiments of the biosensor assay will detect other organisms producing these enzymes, as well as other carbapenemases.

All cited publications are incorporated herein by reference in their entirety.

### EQUIVALENTS

While the invention has been described with respect to illustrative embodiments thereof, it will be understood that various changes may be made to the embodiments without departing from the scope of the invention. Accordingly, the described embodiments are to be considered merely exemplary and the invention is not to be limited thereby.

### REFERENCES

1 Zhong H, Wu M-L, Feng W-J, Huang S-F, Yang P. 2020. Accuracy and applicability of different phenotypic methods for carbapenemase detection in Enterobacteriaceae: A systematic review and meta-analysis. J Glob Antimicrob Resist 21:138-147.
2. Farooqui F, Irfan S, Laiq S M. 2021. Diagnostic Accuracy and Agreement between Four Phenotypic Carbapenemase Detection Tests among Enterobacterales. J Glob Infect Dis 13:133-138.
3. Nordmann P, Poirel L, Dortet L. 2012. Rapid detection of carbapenemase-producing Enterobacteriaceae. Emerg Infect Dis 18:1503-1507.
4. Poirel L, Nordmann P. 2015. Rapidec Carba NP Test for Rapid Detection of Carbapenemase Producers. J Clin Microbiol 53:3003-3008.
5. Tijet N, Boyd D, Patel S N, Mulvey M R, Melano R G. 2013. Evaluation of the Carba NP Test for Rapid Detection of Carbapenemase-Producing Enterobacteriaceae and Pseudomonas aeruginosa. Antimicrob Agents Chemother 57:4578-4580.
6. Iştar EH, Al1şkan HE, Göçmen J S. 2021. Diverse efficacy of CarbaNP test among OXA-48 carbapenemase producing Enterobacterales in an endemic region. Acta Microbiol Immunol Hung 68:34-39.
7. Tijet N, Patel S N, Melano R G. 2016. Detection of carbapenemase activity in Enterobacteriaceae: comparison of the carbapenem inactivation method versus the Carba NP test. J Antimicrob Chemother 71:274-276.
8 Hombach M, von Gunten B, Castelberg C, Bloemberg G V. 2015. Evaluation of the Rapidec Carba NP Test for Detection of Carbapenemases in Enterobacteriaceae. J Clin Microbiol 53:3828-3833.
9. Österblad M, Hakanen A J, Jalava J. 2014. Evaluation of the Carba NP Test for Carbapenemase Detection. Antimicrob Agents Chemother 58:7553-7556.
10. Nordmann P, Sadek M, Demord A, Poirel L. 2020. NitroSpeed-Carba NP Test for Rapid Detection and Differentiation between Different Classes of Carbapenemases in Enterobacterales. J Clin Microbiol 58: e00932-20.
11. Kim J, Kim Y, Abdelazem A Z, Kim H J, Choo H, Kim H S, Kim J O, Park Y-J, Min S-J. 2020. Development of carbapenem-based fluorogenic probes for the clinical screening of carbapenemase-producing bacteria. Bioorganic Chem 94:103405.
12. Ma C-W, Ng K K-H, Yam B H-C, Ho P-L, Kao R Y-T, Yang D. 2021. Rapid Broad Spectrum Detection of Carbapenemases with a Dual Fluorogenic-Colorimetric Probe. J Am Chem Soc 143:6886-6894.
13. Jin S, Lee J Y, Park J Y, Jeon M J. 2020. Xpert Carba-R assay for detection of carbapenemase-producing organisms in patients admitted to emergency rooms. Medicine (Baltimore) 99: e23410.
14. Kaase M, Szabados F, Wassill L, Gatermann S G. 2012. Detection of Carbapenemases in Enterobacteriaceae by a Commercial Multiplex PCR. J Clin Microbiol 50:3115-3118.
15. Wang F, Wang L, Chen H, Li N, Wang Y, Li Y, Liang W. 2021. Rapid Detection of blaKPC, blaNDM, blaOXA-48-like and blaIMP Carbapenemases in Enterobacterales Using Recombinase Polymerase Amplification Combined With Lateral Flow Strip. Front Cell Infect Microbiol 11.
16. Girlich D, Laguide M, Dortet L, Naas T. 2020. Evaluation of the Revogene Carba C Assay for Detection and Differentiation of Carbapenemase-Producing Gram-Negative Bacteria. J Clin Microbiol 58:10.1128/jcm.01927-19.
17. Kamel N A, Tohamy S T, Yahia I S, Aboshanab K M. 2022. Insights on the performance of phenotypic tests versus genotypic tests for the detection of carbapenemase-producing Gram-negative bacilli in resource-limited settings. BMC Microbiol 22:248.
18. Vadlamani G, Thomas M D, Patel T R, Donald L J, Reeve T M, Stetefeld J, Standing K G, Vocadlo D J, Mark B L. 2015. The β-Lactamase Gene Regulator AmpR Is a Tetramer That Recognizes and Binds the d-Ala-d-Ala Motif of its Repressor UDP-N-acetylmuramic Acid (MurNAc)-pentapeptide. J Biol Chem 290:2630-2643.
19. Stachyra T, Péchereau MC, Bruneau J M, Claudon M, Frère J M, Miossec C, Coleman K, Black M T. 2010. Mechanistic studies of the inactivation of TEM-1 and P99 by NXL104, a novel non-beta-lactam beta-lactamase inhibitor. Antimicrob Agents Chemother 54:5132-5138.
20. Papp-Wallace K M, Bethel C R, Distler A M, Kasuboski C, Taracila M, Bonomo R A. 2010. Inhibitor Resistance in the KPC-2 β-Lactamase, a Preeminent Property of This Class A β-Lactamase. Antimicrob Agents Chemother 54:890-897.
21. Livermore D M, Mushtaq S, Warner M, Zhang J, Maharjan S, Doumith M, Woodford N. 2011. Activities of NXL104 combinations with ceftazidime and aztreonam against carbapenemase-producing Enterobacteriaceae. Antimicrob Agents Chemother 55:390-394.
22. Hirvonen V H A, Spencer J, van der Kamp M W. 2021. Antimicrobial Resistance Conferred by OXA-48 β-Lactamases: Towards a Detailed Mechanistic Understanding. Antimicrob Agents Chemother 65: e00184-21.
23. Brem J, van Berkel S S, Zollman D, Lee S Y, Gileadi O, McHugh P J, Walsh T R, McDonough M A, Schofield C J. 2015. Structural Basis of Metallo-β-Lactamase Inhibition by Captopril Stereoisomers. Antimicrob Agents Chemother 60:142-150.
24. Ning N Z, Liu X, Chen F, Zhou P, Hu L, Huang J, Li Z, Huang J, Li T, Wang H. 2018. Embelin Restores Carbapenem Efficacy against NDM-1-Positive Pathogens. Front Microbiol 9:71.
25. Tehrani K, Brüchle NC, Wade N, Mashayekhi V, Pesce D, van Haren M J, Martin N I. 2020. Small Molecule Carboxylates Inhibit Metallo-β-lactamases and Resensitize Carbapenem-Resistant Bacteria to Meropenem. ACS Infect Dis 6:1366-1371.
26. Jackson A C, Zaengle-Barone J M, Puccio E A, Franz K J. 2020. A Cephalosporin Prochelator Inhibits New Delhi Metallo-β-lactamase 1 without Removing Zinc. ACS Infect Dis 6:1264-1272.
27. Performance standards for antimicrobial susceptibility testing. 2001. M100-S11, Clin Microbiol Newsl 23:49.
28. Simner P J, Johnson J K, Brasso W B, Anderson K, Lonsway D R, Pierce V M, Bobenchik A M, Lockett Z C, Charnot-Katsikas A, Westblade L F, Yoo B B, Jenkins S G, Limbago B M, Das S, Roe-Carpenter D E. 2018. Multicenter Evaluation of the Modified Carbapenem Inactivation Method and the Carba NP for Detection of Carbapenemase-Producing Pseudomonas aeruginosa and Acinetobacter baumannii. J Clin Microbiol 56: e01369-17.
29. Tijet N, Boyd D, Patel S N, Mulvey M R, Melano R G. 2013. Evaluation of the Carba NP Test for Rapid Detection of Carbapenemase-Producing Enterobacteriaceae and Pseudomonas aeruginosa. Antimicrob Agents Chemother 57:4578-4580.
30. Iştar EH, Al1şkan HE, Göçmen J S. 2021. Diverse efficacy of CarbaNP test among OXA-48 carbapenemase producing Enterobacterales in an endemic region. Acta Microbiol Immunol Hung 68:34-39.

1. A plasmid comprising:
a reporter;
an inducible promoter;
wherein the inducible promoter enables transcription of the reporter in the presence of one or more products of β-lactam antibiotic activity;
wherein transcription of the reporter produces a signal;
wherein transcription of the reporter in the presence of a β-lactamase produces less signal relative to transcription of the reporter in the presence of a β-lactamase inhibitor.

2. The plasmid of claim 1, wherein the reporter is a luminescent reporter and the signal is luminescence.

3. The plasmid of claim 2, wherein the luminescent reporter comprises a luciferase reporter.

4. The plasmid of claim 3, wherein the luciferase reporter comprises a luxCDABE operon.

5. The plasmid of claim 1, wherein the inducible promoter comprises a carbapenem-inducible ampC promoter and an ampR transcription factor that controls transcription of the ampC promoter.

6. The plasmid of claim 1, having the nucleotide sequence of SEQ ID NO: 1.

7. The plasmid of claim 1, having the nucleotide sequence of SEQ ID NO: 2.

8. A cell transformed with the plasmid of claim 1.

9. The cell of claim 8, wherein the cell is a bacterial cell.

10. The cell of claim 9, wherein the cell is a strain of E. coli.

11. A biosensor cell comprising the plasmid of claim 1.

12. The biosensor cell of claim 11, wherein the reporter is a luminescent reporter and the signal is luminescence.

13. An assay for detecting, assessing, and/or characterizing a β-lactamase inhibitor, comprising:
the biosensor cell of claim 11;
a β-lactam antibiotic;
a β-lactamase; and
a suitable cellular growth medium;
wherein the assay detects, assesses, and/or characterizes the β-lactamase inhibitor added to the assay according to an amount of signal produced by the biosensor cell;
wherein the β-lactamase inhibitor activity is indicated by the biosensor cell producing signal.

14. The assay of claim 13, wherein the β-lactamase is produced by a bacterial cell.

15. The assay of claim 13, wherein the β-lactamase is an isolated enzyme.

16. An assay for detecting, assessing, and/or characterizing a β-lactamase producing organism, comprising:
the biosensor cell of claim 11;
a β-lactam antibiotic;
and
a suitable cellular growth medium;
wherein the assay detects, assesses, and/or characterizes the β-lactamase producing organism added to the assay according to an amount of signal produced by the biosensor cell;
wherein the β-lactamase producing organism is indicated by the biosensor cell producing low or no signal.

17. The assay of claim 16, comprising proteinase K.

18. A kit, comprising:
the biosensor cell of claim 11; and
one or more of a medium that stabilizes the biosensor cell, a β-lactam antibiotic, a β-lactamase, proteinase K, and a suitable cellular growth medium.

19. A method for detecting, assessing, and/or characterizing activity of a compound as a β-lactamase inhibitor, comprising:
incubating the biosensor cell of claim 11 together with a β-lactam antibiotic and a β-lactamase in a suitable cellular growth medium;
adding the compound to the cellular growth medium;
wherein activity of the compound as a β-lactamase inhibitor is detected, assessed, and/or characterized according to an amount of signal produced by the biosensor cell;
wherein the biosensor cell produces signal when the compound exhibits β-lactamase inhibitor activity.

20. The method of claim 19, wherein the growth medium comprises proteinase K.

21. A method for detecting, assessing, and/or characterizing a β-lactamase producing organism, comprising:
incubating the biosensor cell of claim 11 and a β-lactam antibiotic in a suitable cellular growth medium;
adding the organism to the cellular growth medium;
wherein the β-lactamase producing organism is detected, assessed, and/or characterized according to an amount of signal produced by the biosensor cell;
wherein the biosensor cell produces low or no signal when the organism produces the β-lactamase.
